(19) United States
(12) Patent Application Publication
(10) Pub. No.: US 2024/0207253 A1
(43) Pub. Date: Jun. 27, 2024

(54) Inhibitors of CDR1 for Reversal of Azole Drug Resistance in Fungal Infections

(71) Applicants: UNM RAINFOREST INNOVATIONS, Albuquerque, NM (US); THE FLORIDA INTERNATIONAL UNIVERSITY BOARD OF TRUSTEES, Miami, FL (US); THE UNITED STATES GOVERNMENT AS REPRESENTED BY THE DEPARTMENT OF VETERANS AFFAIRS, Washington, DC (US)

(72) Inventors: Larry A. Sklar, Albuquerque, NM (US); Stella Jubay, Albuquerque, NM (US); Clemencia Panilla, Cardiff, CA (US); Marcello Giulianotti, Vero Beach, FL (US); Richard Allen Houghten, Port St. Lucie, FL (US); Samuel Lee, Washington, DC (US)

(21) Appl. No.: 18/283,035

(22) PCT Filed: May 18, 2022
(86) PCT No.: PCT/US2022/029843

§ 371 Date: Sep. 20, 2023

Related U.S. Application Data
(60) Provisional application No. 63/190,628, filed on May 19, 2021.

(51) Int. Cl. A61K 31/496; A61K 31/13; A61K 31/137; A61K 31/155; A61K 31/381; A61K 31/4015; A61K 31/4196; A61K 31/44; A61K 31/495; A61P 31/10

(52) U.S. Cl. CPC A61K 31/496; A61K 31/13; A61K 31/137; A61K 31/155; A61K 31/381; A61K 31/4015; A61K 31/4196; A61K 31/44; A61K 31/495; A61P 31/10

(57) ABSTRACT

The present invention is directed to compounds which have been identified, as inhibitors of the expression of CDR1 (Cdr1p) and/or CDR2 (Cdr2p), both ABC-type plasma, membrane transporters, which are major contributor to antifungal therapy resistance. These compounds may be used in the treatment of fungal infections, especially Candida infections, either alone or in combination with azole antifungal agents such as fluconazole, itraconazole, miconazole, clotrimazole and the like. Often the inhibitor compounds described herein are useful for reversing the antifungal therapy resistance exhibited by the overexpression of CDR1 and/or CDR2 in strains of fungi, especially including Candida spp. Combination therapy and compositions for treating fungal infections, especially including azole resistant fungal infections represent important embodiments of the present invention.

This application claims the benefit of priority of provisional application serial number Ser. No. 63/190,628, filed May 19, 2021 of identical title, the entire contents of which application is incorporated by reference herein.

### RELATED APPLICATIONS AND GRANT SUPPORT

This invention was made with government support under grant no. R21 A1141089 awarded by the National Institute of Health. The government has certain rights in the invention.

### FIELD OF THE INVENTION

The present invention is directed to compounds which have been identified as inhibitors of the expression of CDR1 (Cdr1p), an ABC-type plasma membrane transporter, which is a major contributor to antifungal therapy resistance. These compounds may be used in the treatment of fungal infections, especially Candida infections, either alone or in combination with azole antifungal agents such as fluconazole, itraconazole, miconazole, clotrimazole and the like. Often the CDR1 inhibitors described herein are useful for reversing the antifungal therapy resistance exhibited by the overexpression of CDR1, as well as CDR2. Combination therapy and compositions for treating fungal infections, especially including azole resistant fungal infections are important embodiments of the present invention.

### BACKGROUND AND OVERVIEW OF THE INVENTION

Fungal infections represent a global public health threat. Systemic invasive mycoses are associated with a high mortality rate in immunocompromised individuals; immunosuppression arising from HIV infection, organ transplant and cancer therapy has led to a threefold increase in the incidence of fungal sepsis over the past three decades. These factors, coupled with the rapid spread of resistance to available antifungals, makes novel therapeutic strategies to fight fungi highly desirable.

Candida albicans is a human commensal organism that can cause invasive and disseminated infection in an opportunistic fashion. Candida infections are responsible for increased length of stay, cost of care, and high morbidity and mortality. Each antifungal in the current arsenal is fraught with many limitations and the growing problems of poor response to antifungal therapy and antifungal resistance has underscored the need for new antifungal agents and/or therapeutic approaches to combat fungal infections. The azoles, although usually well-tolerated, are limited by substantial resistance in several clinically relevant non-albicans Candida species. A major contributor to this azole-resistance is over-expression of the ABC-type plasma membrane transporter, Cdr1p. C. albicans genetic mutants lacking CDR1 are hyper-susceptible to fluconazole, and chemical compounds that inhibit Cdr1p functional pump activity can reverse azole drug resistance, though no clinically viable compounds have been developed to date. Therefore, utilizing the novel approach of inhibition of CDR1 (Cdr1p) expression, the present invention is directed to compounds which have exhibited inhibitory activity against CDR1 expression in order to identify agents which may be used as effective therapies, alone or especially in combination with azole antifungal agents in the treatment of fungal infections, especially including Candida fungal infections. These compounds exhibit antifungal activity against fungal growth in vitro, inhibition of CDR1 and transporter expression and in instances antifungal activity, especially including synergistic antifungal activity in combination with a number of azole antifungal agents as otherwise described herein.

### BRIEF DESCRIPTION OF THE INVENTION

The present invention is directed to compounds, compositions and methods which can be used to treat fungal infections, especially including fungal infections which exhibit resistance against azole antifungal agents, especially Candida infections. Pursuant to the present invention, the inventors have discovered a number of CDR1 inhibitor compounds which inhibit CDR1 principally by inhibiting the expression of the ABC plasma membrane transporter CDR1 (Cdr1p), thereby eliminating and/or substantially limiting the ability of a fungus resistant to azole antifungal agents to avoid the therapeutic impact of these agents. These agents also exhibit inhibition of CDR2 (Cdr2p), also an ABC plasma membrane transporter. Compounds according to the present invention may also exhibit antifungal activity, but their principal value is their ability to inhibit the activity of CDR1 and/or CDR2 transporters by limiting and/or eliminating the expression of CDR1 and/or CDR2 plasma membrane transporter, thus rendering previously resistant fungi susceptible to azole antifungal therapy. In embodiments, the compounds according to the present invention, when combined with conventional azole antifungal agents, produce synergistic anti-fungal activity.

In embodiments, the present invention is directed to compounds having the scaffolds 1324, 2222 or 2227 and the compounds which are set forth in FIG. 1, FIG. 2, FIG. 3, FIG. 4, FIG. 5, FIG. 6, FIG. 7, FIG. 8, FIG. 9, FIG. 11, FIG. 12, FIG. 13, FIG. 15, FIG. 16, FIG. 17, FIG. 18, FIG. 19 of the present application. In embodiments the compound is 1324.245, 2222.274, 2227.003, 2227.035, 2227.013 of FIG. 1 or a pharmaceutically acceptable salt, diastereomer, enantiomer, solvate, polymorph or a mixture thereof. In embodiments, the compound is 1324.276, 1324.279, 2220-208, 2220-216, 2220-238, 2222-237, 2222-238, 2222-276, 2227-201 or a pharmaceutically acceptable salt, diasteromer, enantiomer, solvate, polymorph or a mixture thereof. In embodiments, the compound is a compound as set forth in FIGS. 5, 7 and 9, or a pharmaceutically acceptable salt, enantiomer, solvate, polymorph or mixture thereof. In embodiments, the compounds according to the present invention inhibit the expression of CDR1 and in many instances CDR2 and consequently, can be combined with one or more azole antifungal compounds (which set of compounds includes azoles, imidazoles and triazoles) such as voriconazole, itraconazole, posaconazole, ketoconazole, fluconazole, clotrimazole, isavuconazonium, miconozale, econazole, sulconazole, oxiconazole, cyproconazole, defnoconazole, etaconazole, fenbuconazole, flusilazole, hexaconazole, propiconazole, tebuconazole, triademenol, uniconazole, epoxiconazole or a pharmaceutically acceptable salt thereof or a mixture thereof for the treatment of fungal infections, especially drug (azole antifungal) resistant fungal infections, especially including drug resistant Candida spp infections, among other drug resistant fungal species as provided herein below. Preferred azole antifungal agents for use in the present invention include voriconazole, itraconazole, posaconazole, ketoconazole, fluconazole, clotrimazole, isavuconazonium, miconozale, econazole, sulconazole, oxiconazole or pharmaceutically acceptable salts thereof or a mixture thereof. In embodiments, the activity of the combined agents in compositions according to the present invention is synergistic with the CDR1 expression inhibitors and the azole antifungal agents acting in combination to provide an enhanced synergistic (more than additive) effect against fungal infections, especially drug resistant fungal infections.

In embodiments, the CDR1 inhibitor compound used in the present invention is a compound according to the chemical structure:

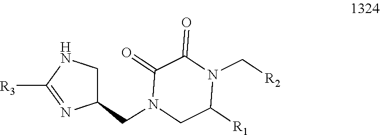

Where R₁ is H, C₁-C₁₂ (often C₁-C₆ or C₁-C₃) alkyl (optionally substituted with 1-3 halogen groups F, Cl, Br or I groups or 1-2 hydroxyl groups), naphthyl, 2-naphthylmethyl, 4-hydroxybenzyl, pyridin-3-yl-methyl, phenyl (optionally substituted with from 1-3 halogen groups or 1-2 hydroxyl groups), benzyl (optionally substituted with from 1-3 halogen groups and/or 1-2 hydroxyl groups) or pyridine-3-yl-methyl (often R₁ groups include S-2-naphthylmethyl, R-2-naphthylmethyl, S-4-hydroxybenzyl, S-pyridin-3-yl-methyl, S-benzyl, gamma-butyl, S-isobutyl, S-propyl, S-2-butyl, R-methyl, R-propyl, R-phenyl, S-tert-butyl, S-isopropyl, S-butyl, R-benzyl, R-2-butyl, R-pyridin-3-yl-methyl, S-4-fluoro-benzyl. R-isopropyl, S-cyclohexyl, R-butyl, S-methyl, R-isobutyl or R-tert-butyl);
R₂ is H, C₁-C₁₂ (often C₁-C₆ or C₁-C₃) alkyl (optionally substituted with 1-3 halogen groups F, Cl, Br or I groups or 1-2 hydroxyl groups) or —(CH₂)phenyl (optionally substituted with 1-3 halogen groups, 1-2 hydroxyl groups, 1-3 —O—C₁-C₃) alkyl groups, and/or 1-3 optionally substituted C₁-C₄ alkyl groups which are optionally substituted with 1-3 halogen or 1-2 hydroxyl groups) (R₂ groups often include 2-(3-bromo-phenyl)-ethyl, m-tolylethyl, 2-(3-fluoro-phenyl)-ethyl, isobutyl, 2-Bicyclo[2.2.1]hept-2-yl-ethyl, cyclohexyl-butyl, cyclobutyl-methyl, n-butyl, ethyl, phenylpropyl,4-methyl-1-cyclohexylmethyl, cycloheptyl-methyl, 2-phenylbutyl, 2-(3,5-bis-trifluoromethyl-phenyl)-ethyl, 2-(4-Isobutyl-phenyl)-propyl, 3-cyclopentyl-propyl, cyclohexyl-methyl, (+/−)-2-Methylbutyl, cyclopentyl-methyl, heptyl, 2-(3-methoxy-phenyl)-ethyl, 4-phenylbutyl, 3-(3,4-dimethoxy-phenyl)-propyl, 3-methylbutyl or phenethyl);
R₃ is H, C₁-C₂₀, often C₁-C₁₂ (often C₁-C₆ or C₁-C₃) alkyl (optionally substituted with 1-3 halogen groups F, Cl, Br or I groups or 1-2 hydroxyl groups), C₂-C₆ alkene, often a vinyl group which is optionally substituted, —(CH₂)ₙphenyl (optionally substituted with 1-3 halogen groups, 1-2 hydroxyl groups, 1-3 —O—C₁-C₃) alkyl groups, and/or 1-3 optionally substituted C₁-C₄ alkyl or alkene groups which are optionally substituted with 1-3 halogen or 1-2 hydroxyl groups), —(CH₂)ₙ-biphenyl (optionally substituted with 1-3 halogen groups, 1-2 hydroxyl groups, 1-3 —O—C₁-C₃) alkyl groups, and/or 1-3 optionally substituted C₁-C₄ alkyl or alkene groups which are optionally substituted with 1-3 halogen or 1-2 hydroxyl groups) with the proviso that R₁, R₂ and R₃, are not each simultaneously H, preferably no more than one of R₁, R₂ and R₃ is H (R₃ groups often include 3-bromo-benzyl, 4-fluoro-phenyl, 4-methyl-cyclohexyl, 3-methyl-phenyl, tert-butyl, Bicyclo[2.2.1]hept-2-yl-methylcyclopentyl, ethyl, cyclobutyl, 4-methoxy-benzyl, 2-cyclohexyl-ethyl, cycloheptyl, propyl, biphenyl-4-yl, 2-(3,4-dimethoxy-phenyl)-ethyl, biphenyl-4-yl-methyl, 3,4-dimethoxy-benzyl, 4-bromo-benzyl, 1-ethyl-pentyl, 2-(2-trifluoromethyl-phenyl)-vinyl, 2-cyclopentyl-ethyl, hexyl, 3-fluoro-benzyl, benzyl or 3-phenyl-propyl; and n is 0-6, often 0-4, or a pharmaceutically acceptable salt, stereoisomer, diastereomer, enantiomer, solvate or polymorph thereof. Alternatively, R₁, R₂ and R₃ groups are set forth in FIG. 5 hereof.

In embodiments, the CDR1 inhibitor is a compound according to the chemical structure:

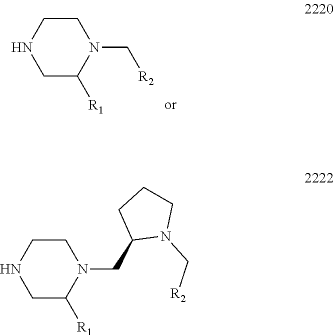

Where R₁ is H, C₁-C₁₂ (often C₁-C₆ or C₁-C₃) alkyl (optionally substituted with 1-3 halogen groups F, Cl, Br or I groups or 1-2 hydroxyl groups), naphthyl, optionally substituted with from 1-3 C₁-C₃ alkyl groups, pyridine, optionally substituted with 1 or 2 C₁-C₃ alkyl groups. —(CH₂)ₙphenyl (optionally substituted with from 1-3 halogen groups, 1-2 hydroxyl groups or 1-3 C₁-C₃ alkyl groups, which are optionally substituted with from 1-3 halogen, often fluoro), (often R₁ is R-3-methylpyridine, S-3-methylpyridine, R-hydroxy-methyl, S-1-hydroxy-ethyl, R-isobutyl, S-hydroxy-methyl, S-4-hydroxybenzyl, R-propyl, R-1-hydroxy-ethyl, S-propanol, S-4-fluorophenyl-methyl, R-methyl, S-methyl, R-butyl, S-isopropyl, R-4-fluorophenyl-methyl, S-propyl, R-2-butyl, R-cyclohexyl-methyl, R-isopropyl, S-2-butyl, S-dimethyl. R-4-chlorophenyl-methyl or R-2-methylnaphthalene);
R₂ is C₁-C₁₂ (often C₁-C₆ or C₁-C₃) alkyl (optionally substituted with 1-3 halogen groups F, Cl, Br or I groups or 1-2 hydroxyl groups), a thiophenyl, furanyl or indolyl group each of which is optionally substituted with from 1-3 C₁-C₃ alkyl groups, —(CH₂)ₙphenyl (optionally substituted with from 1-3 halogen groups, 1-2 hydroxyl groups or 1-3 —O—C₁-C₃ alkyl or 1-3 C₁-C₃ alkyl groups, which are optionally substituted with from 1-3 halogen, often fluoro groups) (R₂ is often 2-thiophene-methyl, m-tolylethyl, 2-(3-methoxy-phenyl)-ethyl, 2-(3-trifluoromethyl-phenyl)-ethyl, 2-methylfuran, 2-(3-fluoro-phenyl)-ethyl, 2-methylbutyl, m-xylene, 4-methyl-benzyl, heptyl, butyl, t-butylmethyl, 4-methoxy-benzyl, (1-phenyl-cyclopropyl)-methyl, p-tolylethyl, 4-ethyoxyphenylethyl, dicyclohexyl-ethyl, 3-indolylethyl 2-(4-methoxy-phenyl)-ethyl, cyclohexyl-ethyl, cyclohexyl-butyl, 3,4-dichlorophenethyl, phenethyl, cyclohexyl-methyl or toluene); and n is 0-6, often 0-4, or a pharmaceutically acceptable salt, stereoisomer, diastereomer, enantiomer, solvate or polymorph thereof. Preferred R₁ and R₂ groups are presented in attached FIG. 7 hereof.

In embodiments, the CDR1 inhibitor is a compound according to the chemical structure:

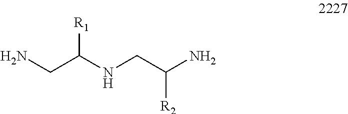

Where R₁ and R₂ are each independently H, C₁-C₁₂ (often C₁-C₆ or C₁-C₃) alkyl (optionally substituted with 1-3 halogen groups F, Cl, Br or I groups, 1-2 hydroxyl groups or an amine group which is optionally substituted with one or two methyl groups), naphthyl optionally substituted with 1-3 C₁-C₃ alkyl groups, pyridine, thiophene or imidazole, each of which is optionally substituted with 1 or 2 C₁-C₃ alkyl groups, C₂-C₆ thioether, naphthyl, optionally substituted with from 1-3 C₁-C₃ alkyl groups, —(CH₂)ₙphenyl (optionally substituted with from 1-3 halogen groups, 1-2 hydroxyl groups, 1-3 —O—C₁-C₃ alkyl groups or 1-3 C₁-C₃ alkyl groups, which are optionally substituted with from 1-3 halogen, often fluoro), analine, optionally substituted with from 1-3 C₁-C₃ alkyl groups, with the proviso that R₁ and R₂ are not both H (often R₁ and R₂ are each independently S-3-(methyl)-propylamine, S-methyl, S-cyclohexyl-methyl, S-4-(methyl)butylamine, R-3-methylpyridine, dimethyl, R-2-methylnaphthalene. R-methyl-imidazolyl, S-isopropyl, R-isobutyl, R-2-methylthiophene, S-propyl, R-propyl, S-butyl, S-2-methylthiophene, R-1-hydroxy-ethyl, R-isopropyl, R-2-(methylthio)ethyl, S-4-ethoxybenzyl, R-2-butyl, R-methyl, R-4-methylaniline, R-4-ethoxybenzyl, S-ethyl or R-ethanol; and n is 0-6, often 0-4, or a pharmaceutically acceptable salt, stereoisomer, diastereomer, enantiomer, solvate or polymorph thereof. In embodiments preferred R₁ and R₂ substituent groups are set forth in FIG. 9 hereof.

In embodiments, each of the preferred substituents for R₁, R₂ and R₃ are set forth in each of FIGS. 5, 7 and 9 with respect to compound 1324 (FIG. 5), compounds 2220 and 2222 (FIG. 7) and compound 2227 (FIG. 9). These FIGURES provide preferred substituent groups based upon analyses described in the experimental sections.

In embodiments, the present invention is directed to pharmaceutical compositions comprising an effective amount of a CDR1 inhibitor compound as described above and in the FIGURES and examples described herein, alone or often in combination with an azole antifungal agent(s). In embodiments, the azole antifungal agent is often voriconazole, itraconazole, posaconazole, ketoconazole, fluconazole, clotrimazole, isavuconazonium, miconozale, econazole, sulconazole, oxiconazole or other azole (azole, imidazole or triazole) antifungal agent or a pharmaceutically acceptable salt thereof or a mixture thereof. In embodiments, the antifungal agent is voriconazole, itraconazole, posaconazole, ketoconazole, fluconazole, clotrimazole, isavuconazonium, miconozale, econazole, sulconazole, oxiconazole, cyproconazole, defnoconazole, etaconazole, fenbuconazole, flusilazole, hexaconazole, propiconazole, tebuconazole, triademenol, uniconazole, epoxiconazole or a pharmaceutically acceptable salt or mixture thereof. In embodiments, the pharmaceutical composition often comprises an effective amount of at least one CDR1 inhibitor compound or a pharmaceutically acceptable salt thereof in combination with at least one azole antifungal agent in combination with a pharmaceutically acceptable carrier, additive or excipient.

In embodiments, the present invention is directed to a method for treating a fungal infection, often a drug resistant fungal infection comprising administering a CDR1 inhibitor as described herein to a patient or subject in need. In embodiments, the present invention is directed to inhibiting, treating and/or eliminating fungal infections and/or azole antifungal drug resistance in a patient or subject in need, the method comprising administering to the patient or subject an effective amount of a compound presented herein as a CDR1 inhibitor and often a CDR2 inhibitor as described herein. In embodiments, the method for treating fungal infections comprises co-administering an effective amount of a CDR1 inhibitor as described herein and an azole (azole, imidazole and/or triazole) antifungal agent to a patient in order in a single composition or separately to effect antifungal therapy. In embodiments, the combination of a CDR1 inhibitor and an azole antifungal agent is synergistic in its inhibitory and/or therapeutic effect on fungal infections, especially including azole antifungal drug resistant fungal infections such as Candida spp. and other resistant fungal infections as otherwise described herein, among others.

These and/or other embodiments and aspects of the invention may be readily gleaned from a review of the detailed description of the invention that follows.

### BRIEF DESCRIPTION OF THE FIGURES

FIG. 1 shows compounds identified herein as 1324.245, 2222.274, 2227.003, 2227.035 and 2227.013 as having excellent activity as CDR1 inhibitors for use in the present invention. The first part (XXXX) of the compound names (XXXX.YYY) indicates the scaffold from which the compounds is based and the second part (YYY) indicates that these refer to single compounds, as opposed to mixtures of compounds from the same scaffold. Scaffold and function R-groups are discussed infra. Additional compounds shown as 1324.276, 1324.279, 2220.208, 2220.216, 2220.238, 2222.237, 2222.238, 2222.276 and 2227.201, also exhibit substantial potential CDR1 inhibitors.

FIG. 2 shows Table 1, which provides a summary of characteristics of compounds 1324.245, 2222.274, 2227.003, 2227.035 and 2227.013 which are presented in FIG. 1, described above. These compounds show excellent CDR1 specificity, effective synergy with fluconazole, demonstrable anti-fungal activity which may be generalized to different fungal species and low cytotoxicity to mammalian cells.

FIG. 3 shows the eighteen scaffold ranking samples chosen for dose response studies set forth in Table 2.

FIG. 4 shows a summary of identified scaffolds. Scaffolds that were advanced to the positional scan screening as well as some analog scaffolds that showed some efficacy in the scaffold dose response study. A. Shows the 4 scaffolds advanced to positional scanning: 1324, 2220, 2222, 2227. (B) Shows analogs of the scaffolds selected that also showed activity in the scaffold screen: 1664, 1666, 1665, 2161. The blue shaded scaffolds are all piperazines and the green shaded ones are all polyamines.

FIG. 5 shows scaffold 1324 and functionalities at each of the R positions that are incorporated in the library. The entire library contains 56,640 individual compounds formatted into 116 samples. The relative response ratios of each sample are shown by the color bars (scale of green/light gray at the top of each column represents 100 & to red/dark gray at bottom of each column represents 0% at 25 μg/ml); samples are ordered in each position by relative response ratios.

FIG. 6 shows the SAR from scaffold 1324. Shown are the positions of three R groups and conclusions regarding functionalities as supported by data from screening the 1324 Position Scanning Library.

FIG. 7 shows functionalities of R groups on scaffolds 2220 and 2222. Scaffold 2220 (top) and 2222 (right) have the same 39 R₁ functionalities and 60 R₂ functionalities (table right). Each at each of the R positions that are incorporated in the library. The entire library contains 56,640 individual compounds formatted into 116 samples. The relative response ratios of each sample are shown by the color bars (scale of green/light gray at top of each column represents 100 & to red/dark gray at bottom of each column represents 0% at 25 μg/ml); samples are ordered in each position by relative response ratios of 2222.

FIG. 8 shows the SAR from scaffolds 2220 and 2222. Shown are the positions of R groups present on scaffold 2220 (left) and 2222 (right) with conclusions regarding functionalities as supported by data from screening the respective Positional Scanning Library.

FIG. 9 shows scaffold 2227 and functionalities at each of the R positions that incorporated in the library. Scaffold 2227 has the same 60R1 and R2 functionalities (table right). Each library contains 3,600 individual compounds formatted into 120 samples. The relative response ratios of each sample are shown by the color bars (scale of green/light gray at top of each column represents 100% to red/dark gray at bottom of each column represents 0% at 12.5 μg/ml); samples are ordered in each position by relative response ratios.

FIG. 10 shows the density of Cdr1p at the plasma membrane. ImageJ was used to estimate the Corrected Total Cell fluorescence of Cdr1p-GFP at the plasma membrane which was expressed in relation to the estimated surface area of the cell. The dot plot shows the calculated density of Cdr1p on the surface of the cell from at least 100 cells per condition, with the Geometric Mean values (colored heavy horizontal line) and the standard deviation of the geometric mean (error bars) indicated.

FIG. 11 shows in Table 3 that TPI compounds can increase fluconazole sensitivity in azole-resistant isolates of varying species of Candida.

FIG. 12 shows the results of a fluconazole-TP12227.035 combination antifungal susceptibility assay. (A) shows the chemical structure of the top compounds. (B) the top panels present heatmaps of assay plates testing all possible combinations of TPI 2227.035 (from 90 μM) and fluconazole (from 64 μg/ml). The top rows of each plate carry only fluconazole in 2-fold dilutions. The heatmap indicates the proportion of cell death in each well, compared to untreated control, in a scale ranging from 0% (black) to 100% (white). The bottom panels have the corresponding Synscreen 3D plots of checkerboard assays presented above, where the surface plotted represents the theoretical additive threshold based on the effect on growth of each individual compound. The z-axis represents % Reduction in growth relative to non-treated control. Red pin-heads that lay above the surface of the plot are points at which synergistic drug interaction is predicted. Data from the average of three independent, biological replicate experiments are shown.

FIG. 13 shows Table A1. This table evidences that the TPI compounds listed do not exhibit general antifungal properties.

FIG. 14 shows representative FL1 (green channel) and #Events histograms comparing Median Channel Fluorescence of samples 5674Con and SolCon. Shown are representative flow cytometry histograms displaying the distribution of each sample population (n=16 wells per sample) in terms of GFP levels (FL1) vs Number of Events. The averaged Median Channel Fluorescence (MCF) and standard deviation for each population are indicated. The Response Ratio (RespRatio) value is the MCF of that sample with respect to the MCF of untreated control wells (5674Con). These results demonstrate that 0.5% (v/v) of the dimethylformamide solvent (SolCon) does not result in modulation of Cdr1p-GFP levels.

FIG. 15 shows Table A2. This table evidences the cytotoxicity profile of certain TPI compounds as indicated.

FIG. 16 shows the IC50-tox histograms for the indicated TPI compounds.

FIG. 17 shows the results of a Fluconazole-TP12227.003 combination antifungal susceptibility assay. (A) The top panels present heatmaps of assay plates testing all possible combinations of TPI 2227.003 (from 90 μM) and fluconazole (from 64 μg/ml). The top rows of each plate carry only fluconazole in 2-fold increases, left to right. The first columns of each plate carry only TP12227.003 in 2-fold increases, top to bottom. The heatmap indicates the proportion of cell death in each well, compared to untreated control, in a scale ranging from 0% (black) to 100% (white). The bottom panels are the corresponding Synscreen 3D plots of checkerboard assays presented above, where the surface plotted represents the theoretical additive threshold based on the effect on growth in the presence of each of the two individual compounds. The z-axis represents % Reduction in growth relative to non-treated control. Red pin-heads that lay above the surface of the plot are points at which synergistic drug interaction is predicted. Data from the average of three independent, biological replicate experiments are shown. (B) Shows the chemical structure of TPI 2227.003.

FIG. 18 shows the results of a Fluconazole-TPI2227.013 combination antifungal susceptibility assay. (A) The top panels present heatmaps of assay plates testing all possible combinations of TPI 2227.013 (from 62 μM) and fluconazole (from 64 μg/ml). The top rows of each plate carry only fluconazole in 2-fold increases, left to right. The first columns of each plate carry only TPI2227.013 in 2-fold increases, top to bottom. The heatmap indicates the proportion of cell death in each well, compared to untreated control, in a scale ranging from 0% (black) to 100% (white). The bottom panels are the corresponding Synscreen 3D plots of checkerboard assays presented above, where the surface plotted represents the theoretical additive threshold based on the effect on growth in the presence of each of the two individual compounds. The z-axis represents % Reduction in growth relative to non-treated control. Red pin-heads that lay above the surface of the plot are points at which synergistic drug interaction is predicted. Data from the average of three independent, biological replicate experiments are shown. (B) shows the chemical structure of TPI 2227.013.

FIG. 19 shows that TPI compounds downregulate CDR1 and CDR2 expression at the gene level. Expression profiles of targets genes were assessed using RT-qPCR in matched clinical isolates 5457, a fluconazole-sensitive isolate (FLC^{S}), and the fluconazole-resistant isolate (FLC^{R}) 5674 that emerged from 5457. Results shown are from triplicate reactions from three biological replicates of each condition tested. Significant differences in relative gene expression were tested using the unpaired t-test (Prism 9.0, GraphPad) and asterisks indicate statistical significance, p<0.05.

FIG. 20 shows that TPI2227.013 possesses a wide therapeutic window. Human hepatic cell line HepG2 were incubated with serial 2-fold dilutions of the compound at a starting concentration of 250 μg/ml. Following 24 h of co-incubation, viability of the cells were assessed using an XTT assay. The concentration at which 50% of cells are killed was estimated using the variable-slope, normalized response non-linear fit curve (Prism 9.3, GraphPad).

FIG. 21 shows that TP12227.013 does not have general antifungal activity. Clinical isolate strain 5674 was incubated with varying concentrations of TP12227.013 and cell density was followed over 30 hours to assess the compound's effect on C. albicans growth. Growth rates (doubling times) for each condition was calculated from the exponential phase of growth and compared to the untreated controls where no compound was added.

### DETAILED DESCRIPTION OF THE INVENTION

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. The term “about” is used in context to describe a value or range of values which is representative of the literal value of a number or numbers within a range up to ±10% of the value or range of values described.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a.” “and” and “the” include plural references unless the context clearly dictates otherwise.

Furthermore, the following terms shall have the definitions set out below.

The term “patient” or “subject” is used throughout the specification within context to describe an animal, generally a mammal and preferably a human, to whom treatment, including prophylactic treatment (prophylaxis), of fungal infections according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a CDR1 inhibitor (which often also functions as a CDR2 inhibitor) and/or an azole antifungal agent which, in context, are used to produce or effect an intended result, whether that result relates to the prophylaxis and/or therapy of fungal infections, especially drug resistant fungal infections as otherwise described herein. The term effective subsumes all other effective amount or effective concentration terms (including the term “therapeutically effective”) which are otherwise described or used in the present application.

The term “compound”, as used herein, unless otherwise indicated, refers to any specific chemical compound disclosed herein and includes tautomers, regioisomers, geometric isomers, stereoisomers and where applicable, optical isomers (diastereomers, enantiomers) thereof, as well as pharmaceutically acceptable salts and derivatives (including prodrug forms) thereof. Within its use in context, the term compound generally refers to a single compound, but also may include other compounds such as stereoisomers, regioisomers and/or optical isomers/enantiomers (including racemic mixtures) as well as specific enantiomers or enantiomerically enriched mixtures of disclosed compounds. The term also refers, within context, to prodrug forms of compounds which have been modified to facilitate the administration and delivery of compounds to a site of activity. It is noted that in describing the present compounds, numerous substituents and variables associated with same, among others, are described. The use of a bond presented as [symbol] signifies that a single bond is present or absent, depending on the context of the chemistry described. The use of a bond presented as [symbol] signifies that a single bond or a double bond is intended depending on the context of the chemistry described. It is understood by those of ordinary skill that molecules which are described herein are stable compounds as generally described hereunder.

The term “effective” is used herein, unless otherwise indicated, to describe an amount of a compound or composition which, in context, is used to produce or effect an intended result, whether that result relates to the inhibition of the effects of a disease state (e.g. a fungal infection and/or condition) on a subject or the treatment or prophylaxis of a subject for secondary conditions, disease states or manifestations of disease states as otherwise described herein. This term subsumes all other effective amount or effective concentration terms (including the term “therapeutically effective”) which are otherwise described in the present application.

The term “CDR1 inhibitor” or “CDR1 inhibitor compound” is used to describe a compound that inhibits the expression of CDR1 and/or CDR2, principally CDR1. In certain instances, the compound may also exhibit anti-fungal activity, but in general, the principal utility of CDR1 inhibitors in the present invention serves to inhibit expression of CDR1 and/or CDR2, which increases the susceptibility of the fungus to an azole antifungal agent to which the fungus has become resistant as otherwise described herein.

The terms “treat”, “treating”, and “treatment”, etc., as used herein, refer to any action providing a benefit to a patient at risk for a fungal infection, including improvement in the condition through lessening or suppression of at least one symptom, inhibition of fungal growth, reduction in fungal cells, prevention, reduction in the likelihood or delay in progression of the spread of a fungal infection, prevention or delay in the onset of disease states or conditions which occur secondary to fungal infections, among others. Treatment, as used herein, encompasses both prophylactic and therapeutic treatment, within context. The term “prophylactic” when used, means to reduce the likelihood of an occurrence or the severity of an occurrence (including the spread of a fungal infection) within the context of the treatment of a fungal infection, including a fungal infection as otherwise described hereinabove.

The term “fungus” or “fungus infection” is used to describe any of a diverse group of eukaryotic single-celled or multinucleate organisms or an infection caused thereby that live by decomposing and absorbing the organic material in which they grow. Fungi pursuant to the present invention comprise mushrooms, molds, mildews, smuts, rusts, and yeasts, for purposes of the present invention principally molds and yeasts and classified in the kingdom Fungi or, in some alternative classification systems, in the division Thallophyta of the kingdom Plantae, which cause infections. In embodiments, the fungus is a member of Candida spp., including C. albicans, C krusei. C. rugosa, C. glabrata, C. parapsilosis, C. tropicalis, C. dubliniensis and C. auris, among others, including Aspergillus spp. many of which strains are resistant to azole antifungal agents. In embodiments, the fungus is a member of Tricophyton spp., Histoplasma spp., Blastomyces spp., Paracoccidioides spp., Cryptococcus spp., Pneumocystis spp. (especially P. jirovecii). Mucor spp., Taloromyces spp., Sporothrix spp., Rhizopus spp., and Absidia spp. among others. The term “azole antifungal agent resistant fungal infection”, “azole resistant antifungal infection” or resistant antifungal infection” is a fungal infection which has developed resistance to an antifungal agent, in particular an azole antifungal agent as otherwise described herein.

Fungal infections which may be treated by compounds and/or compositions according to the present invention include, for example, dermatological fungal diseases and/or conditions, respiratory fungal diseases and/or conditions, neurological fungal diseases and/or conditions and hepatic fungal diseases and/or conditions, especially including those fungal infections which have exhibited resistance to azole antifungal agents. Dermatologic fungal diseases and/or conditions are caused principally by a group of fungi commonly referred to as dermatophytes, including for example. Tinea versicolor (caused by P. orbiculare, or P. ovale), Athlete's Foot (Tinea pedis), Jock Itch (Tinea cruris), Ringworm of the body (Tinea corporis), Tinea of the beard (Tinea barbae) and Tinea of the scalp (Tinea capitis), among others. Respiratory fungal diseases and/or conditions and the fungus which is the infective agent for such diseases and/or conditions include Histoplasmosis (H. capsulatum), Blastomycosis (B. dermatitidis), Coccidiodomycosis (C. immitis, or Coccidiodes posadasti), Paracoccidiodomycosis (Paracoccidioides brasiliensis), Cryptococcosis (Cryptococcus neoformans, or Cryptococcus gattii), Aspergillosis (Aspergillus spp), Zygomycosis (caused by members of the genera Mucor, Rhizopus, or Absidia), Candidiasis (C. albicans, C. tropicaahs, or C. glabrata), Pneumocystis pneumonia (Pneumocystis jirovecii), Rhizopus spp. Fungal respiratory diseases and/or conditions range in severity from asymptomatic, to presentation with mild malaise, to life threatening respiratory disease. Neurological fungal diseases and/or conditions including for example meningitis (caused by Cryptococcus spp, Aspergillus spp., Pseudallescheria boydii, Coccidiodies spp., Blastomyces dermatitidis, and Histoplasma capsulatum) and Brain Abscess (caused by Candida spp. Aspergillus spp. Rhizopus spp. Mucor spp. P. boydii) often seen in immunocompromised individuals. Hepatic fungal diseases and/or conditions and agents which cause such diseases and/or conditions. include Histoplasmosis (Histoplasma capsulatum) and Candidiasis (Candida spp).

The term “additional anti-fungal agent” is used to describe a traditional or non-traditional anti-fungal agent other than an azole antifungal agent which can be combined with compounds and/or compositions according to the present invention either in a single composition or as a co-administered combination in treating fungal infections pursuant to the present invention. Additional anti-fungal agents include, for example, the polyenes, allylamines, and echinocandins, as well as miscellaneous anti-fungal agents. Polyene antifungals include nystatin and amphotericin. Allylamines include terbinafine. Echinocandins include anidulafungin, caspofungin and micafungin. Miscellaneous anti-fungal agents include flucytosine, griseofulvin and pentamine. Non-traditional anti-fungal agents such as oil of oregano, tea tree oil, caprylic acid, tumeric/curcumin, and the like may also be included in compositions and methods according to the present invention.

The term “pharmaceutically acceptable salt” is used throughout the specification to describe a salt form of one or more of the compounds herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be particularly preferred as neutralization salts of carboxylic acid containing compositions according to the present invention. The term “salt” shall mean any salt consistent with the use of the compounds according to the present invention. In the case where the compounds are used in pharmaceutical indications, including the treatment of fungal infections, the term “salt” shall mean a pharmaceutically acceptable salt, consistent with the use of the compounds as pharmaceutical agents.

The term “alkyl” refers to a fully saturated monovalent radical containing carbon and hydrogen, and which may be cyclic (including bicyclic), branched or a straight chain containing from 1 to 20 carbon atoms, often 1-12 carbon atoms (1, 2, 3, 4, 5, 6, 7, 8, 9, 10, 11 or 12), preferably 1, 2, 3, 4, 5 or 6 carbon atoms. Examples of alkyl groups are methyl, ethyl, n-butyl, n-hexyl, n-heptyl, n-octyl, isopropyl, 2-methylpropyl, cyclopropyl, cyclopropyl-methyl, cyclobutyl, cyclopentyl, cyclopentylethyl, cyclohexylethyl and cyclohexyl, among others. Preferred alkyl groups are C₁-C₆ or C₁-C₃ alkyl groups. Where used, the term C₀ refers to a hydrogen substituent H. “Alkylene” (e.g., methylene) when used, refers to a fully saturated hydrocarbon which is divalent (may be linear, branched or cyclic) and which is optionally substituted. Other terms used to indicate substitutent groups in compounds according to the present invention are as conventionally used in the art. Aryl groups refer to aromatic carbocyclic or heterocyclic groups. Preferred aryl groups are phenyl groups or naphtyl which are optionally substituted. Preferred aromatic heterocyclic (heteroaryl) groups are 5-6 membered rings having at least one S, O, N or P atom in the ring or groups including indolyl.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. CDR1 inhibitor compounds according to the present invention often are administered with one or more azole antifungal agents and/or additional anti-fungal agents or other agents which are used to treat and/or ameliorate the symptoms of fungal infections. Exemplary additional anti-fungal agents which may be coadministered in combination with CDR1 inhibitors and azole antifungal agents (e.g. azoles, imidazoles, triazoles) include, for example, polyenes, allylamines, and echinocandins, as well as miscellaneous agents, among numerous others, as otherwise described herein. In certain aspects of the present invention, compositions according to the invention which include an effective amount of a CDR1 inhibitor and an azole antifungal agent are co-administered with echnocandins, which are favorably administered to trigger a specific drug-induced condition by reducing the amount of glucan in the fungal cell wall and increasing the synthesis and surface exposure of chitin, making the fungus more vulnerable to inhibition. The result is often a favorable impact on fungal growth.

The compositions of the present invention may be administered orally, parenterally, by inhalation spray, topically, rectally, nasally, buccally, vaginally or via an implanted reservoir or by any other traditional administrative regin. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. In embodiments, the composition is employed as a topical ointment, cream or lotion, preferably in a combination composition for topical administration. Transdermal formulations are also contemplated by the present invention.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically and in embodiments, this may be a preferred administration route. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. In certain preferred aspects of the invention, the topical cream or lotion may be used prophylactically to prevent infection when applied topically in areas prone toward virus infection. In additional aspects, the compounds according to the present invention may be coated onto the inner surface of a condom and utilized to reduce the likelihood of infection during sexual activity.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of a CDR1 inhibitor compound in a pharmaceutical composition of the instant invention that may be combined with the carrier materials to produce a single dosage form is an effective amount which will vary depending upon the host and fungal disease or infection treated and the particular mode of administration. Preferably, the compositions should be formulated to contain between about 0.05 milligram to about 1 to several grams, more preferably about 1 milligram to about 750 milligrams, and even more preferably about 10 milligrams to about 500-600 milligrams of the CDR1 inhibitor active ingredient alone or in embodiments, in combination with at least one azole antifungal agent and optionally an additional antifungal agent or other bioactive agents useful in the treatment of fungal infections or symptoms associated with fungal infections all in effective amounts. The amount of each agent used is an effective amount, generally within the amounts which are presented herein above. In embodiments, the amount of CDR1 and azole antifungal agents produces a synergistic effect (e.g. inhibition of fungal growth, fungicidal action, etc.) on the fungal infection treated. In embodiments, CDR1 inhibitors which also exhibit substantial CDR2 inhibition may also exhibit greater activity in treating fungal inventions in combination with azole antifungal agents as described herein than those compounds which are principally selective for CDR1 inhibition.

It should also be understood that a specific dosage and treatment regimen for any particular patient will vary depending upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject (e.g. a male or female human) suffering from a fungal infection, especially including a drug (azole antifungal agent) resistant fungal infection, can be treated by administering to the patient (subject) an effective amount of the CDR1 inhibitor compound according to the present invention including pharmaceutically acceptable salts, solvates or polymorphs, thereof in a pharmaceutically acceptable carrier or diluent, either alone, or preferably in combination with other known pharmaceutical agents, especially at least one azole antifungal agent or another agent which can assist in treating the fungal infection or ameliorate the secondary effects and conditions associated with the fungal infection. This treatment can also be administered in conjunction with other conventional antifungal therapy.

These compounds and/or compositions can be administered by any appropriate route, for example, orally, parenterally, intravenously, intradermally, subcutaneously, or topically, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably about 0.05-0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, preferably about 5 to 500-600 mg or more of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel. or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound(s) or pharmaceutically acceptable salts thereof can also be mixed with other active materials that do not impair the desired action, or with materials that supplement the desired action, such as other antifungal agents (i.e., antifungal agents to which the treated fungal infection is not resistant), anti-HIV agents, antibiotics, anti-inflammatories, or antiviral compounds. In certain embodiments, one or more CDR1 inhibitor compounds according to the present invention is coadministered with at least one additional azole antifungal agent and at least one additional antifungal agent in the treatment of a fungal infection.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

### EXAMPLES

Experiments for identifying CDR1 inhibitor compounds for use in the present invention are presented in the experiments detailed herein.

### Examples

Candida albicans can cause invasive and disseminated infection in an opportunistic fashion and Candida infections are responsible for increased length of stay, cost of care, and high morbidity and mortality. At present, the antifungal agents used to treat these and other fungal infections have many limitations and the growing problems of poor response to antifungal therapy and antifungal resistance has underscored the need for new antifungal agents and/or therapeutic approaches to combat fungal infections. The azoles, although usually well-tolerated, are limited by substantial resistance in several clinically relevant non-albicans Candida species. A major contributor to this azole-resistance is over-expression of the ABC-type plasma membrane transporter, Cdr1p. C. albicans genetic mutants lacking CDR1 are hyper-susceptible to fluconazole, and chemical compounds that inhibit Cdr1p functional pump activity can reverse azole drug resistance, though no clinically viable compounds have been developed to date. The task of the present invention was to identify compounds which inhibit CDR1 and/or CDR2 and could be used in combination with antifungal agents, in particular, azole antifungal agents, to enhance antifungal therapy by inhibiting resistance to these agents by various species of fungi.

Utilizing the novel approach of inhibition of CDR1 (Cdr1p) expression, initial experiments were directed to screening libraries of small molecules using a high-throughput whole cell fluorescence-based flow cytometry assay to identify agents that inhibit expression of CDR1-GFP in an azole-resistant C. albicans strain. These positive hits were then further tested using secondary assays for in vitro growth, antifungal activity in combination with fluconazole, and target-specific transporter expression.

To that end, the project completed the screen of the Torrey Pines Molecular Library which is composed of 79 combinatorial scaffolds representing >30 million compounds. Scaffolds that caused a significant reduction in Cdr1p-GFP levels and considered positive hits were then ranked by dose-response data. Top scaffold libraries were next further structurally assessed using a positional scanning screening method that allowed for the identification of features that provide activity specific for each scaffold. Moreover, the positional scanning screen allowed for structure activity relationship (SAR) discovery that provides the basis for future chemical optimization. Further, single compounds that effected the desired outcome in a dose-responsive manner, with an EC₅₀ of 10 μM or better, were moved forward into further experiments.

Further experiments were directed to completing the bulk of the secondary assays, as well as pursuing additional work on promising hits. Each compound was assessed for general antifungal activity using growth assays from which the effect of hit compound on growth rate was determined. Coupled with assays designed to identify compounds with antifungal properties causing cell wall and/or plasma membrane damage, the experiments demonstrated that the effect of hit compounds on CDR1 expression levels was not the indirect result of fungistatic or fungicidal properties of hit compounds. To further gain insight into possible mechanisms of action, assessment of the effect of each compound on CDR1 mRNA levels (qRT-PCR) and Cdr1p expression at the plasma membrane (fluorescence microscopy) was conducted. Trends from the qRT-PCR suggested that the compounds act on mechanisms that result in the downregulation of gene expression of CDR1 (and CDR2). Results from measuring Cdr1p-GFP at the plasma membrane using fluorescence microscopy techniques further provide support to the hypothesis that the compounds act to reduce Cdr1p expression at the plasma membrane where it functions. As azole-resistance is related to the levels of overexpression of Cdr1p on the plasma membrane, it was reasoned that hit compounds that effectively lower Cdr1p levels will increase fluconazole susceptibility in azole-resistant isolates. It was shown that at least one compound from each of the unique scaffolds identified in our screen had synergistically increased the potency of fluconazole against our clinical isolate 5674 and have prioritized them accordingly. Further, it was demonstrated that these have potential for use against non-albicans azole-resistant isolates (C. glabrata, C parapsilosis, and C. krusei). Lastly, the inventors assessed the safety of each hit compound in mammalian cytotoxicity assays and together with pharmacological data of each compound, these results strongly suggest therapeutic safety and potential, and marked flexibility for drug optimization. All screening, dose-response, and secondary assays were performed in triplicate, using independent biological replicates, to ensure results were unbiased and reproducible. Statistical evaluation of results were performed and appropriate based on the experimental design.

In summary, the data collected and presented herein support the present invention and not only evidence the effectiveness of the present invention but further demonstrate the potential clinical utility of compounds according to the present invention with tests for generalizability to a number of other fungal species. In addition, the inventors synthesized a small group of compounds based on SAR data which were put through the workflow; the outcome of this endeavor added to the list of compounds useful in the present invention. Overall, the results demonstrate that the compounds identified have a substantial potential to increase the clinical utility of well-tolerated azoles by increasing or restoring azole-susceptibility in azole-resistant strains, irrespective of Cdr1p involvement in the mechanism of azole-resistance (e.g. C. krusei). The compounds themselves possess good biological activity with drug-like properties that allows for extensive opportunities for modifying their structures to improve activity, thus further increasing the likelihood of moving a drug forward into pre-clinical IND studies.

### Primary Screen

In this set of experiments, the inventors primarily focused on data generated from the use of the Torrey Pines Molecular Library. The Torrey Pines Molecular Library (TPML), developed by the Torrey Pines Institute for Molecular Studies (TPIMS now FIU-TPIMS) is a combinatorial library of over 30 million traditional small molecules and peptides that are selected for unique chemical and pharmacological diversity and a range of biological activities. The library design allows for the screen of the entire collection by testing exponentially fewer samples in order to identify individual hits. The diversity of the TPML has been characterized and described quantitatively by means of molecular scaffolds, molecular properties, and structural fingerprints. Additional studies evaluating medicinally relevant chemical space demonstrate that the TPML collection contains a larger degree of molecular complexity and 3D conformational space (often calculated by the fraction of sp3 orbitals, F-sp) than readily available commercial collections. Fingerprint-based similarity studies demonstrate that within the library there are dense regions in chemical space. The high-density coverage increases the potential of identifying activity cliffs and provides a rapid understanding of the SAR associated with novel hits and targets. The hits identified are thus amenable to lead optimization.

### Scaffold Ranking

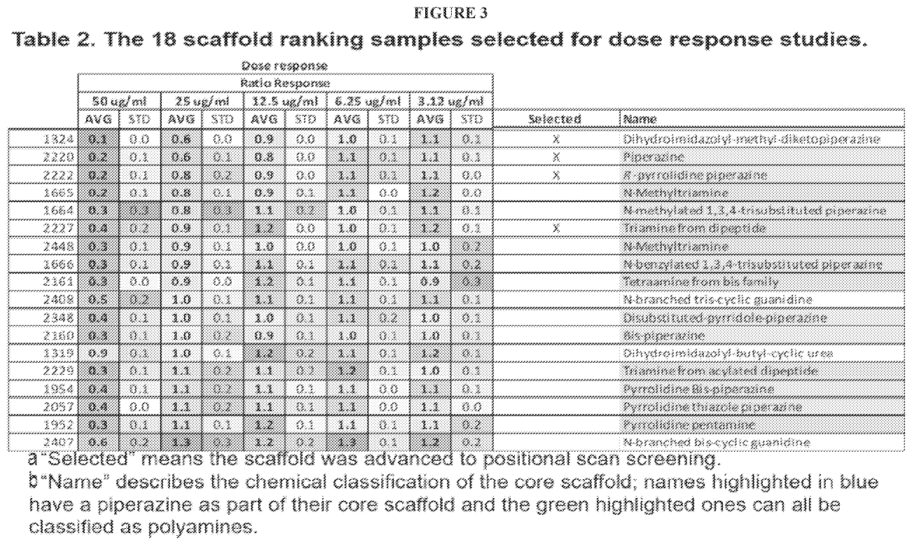

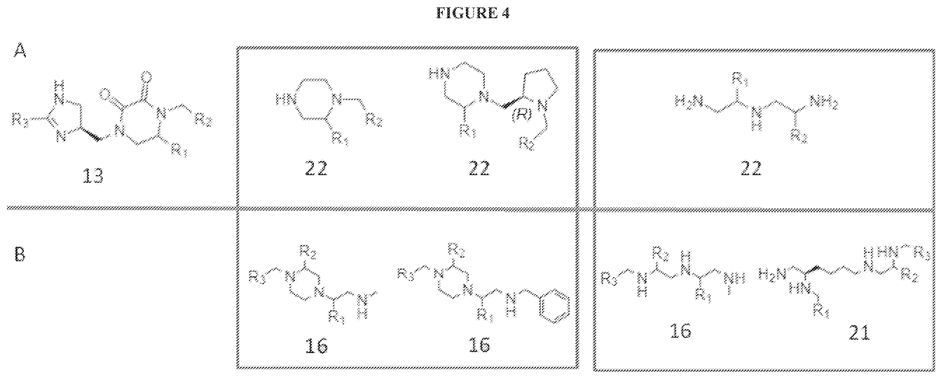

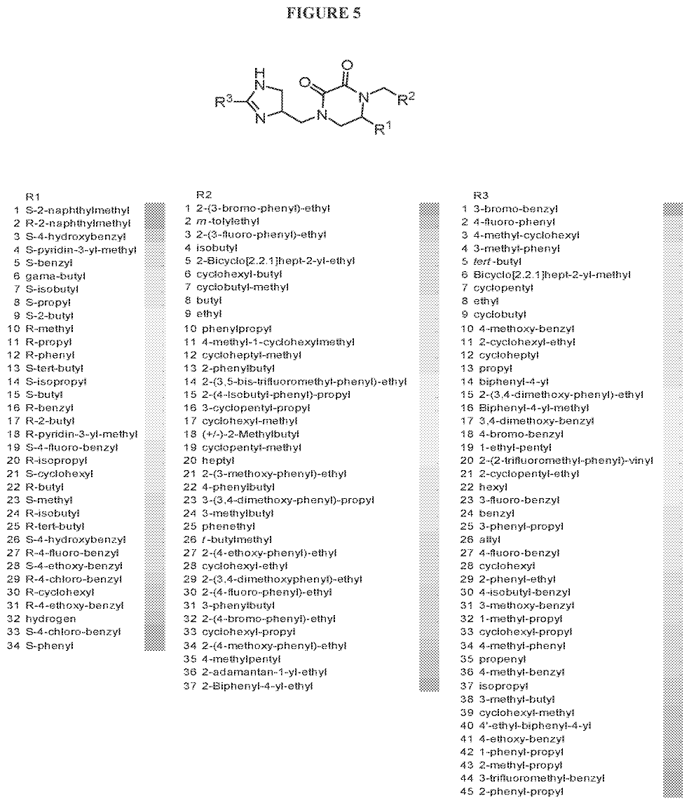

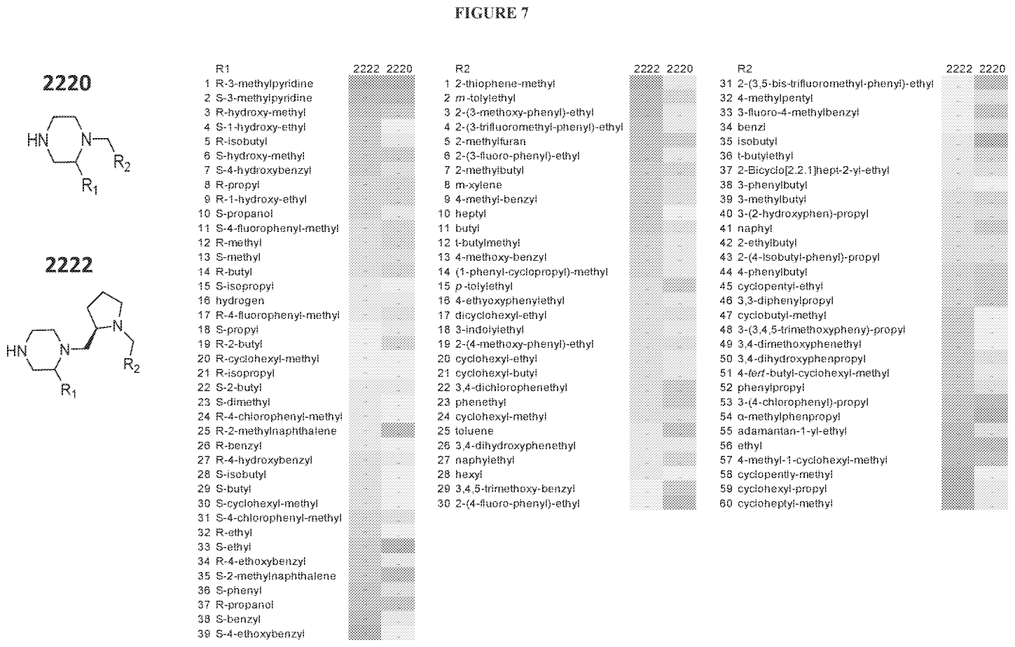

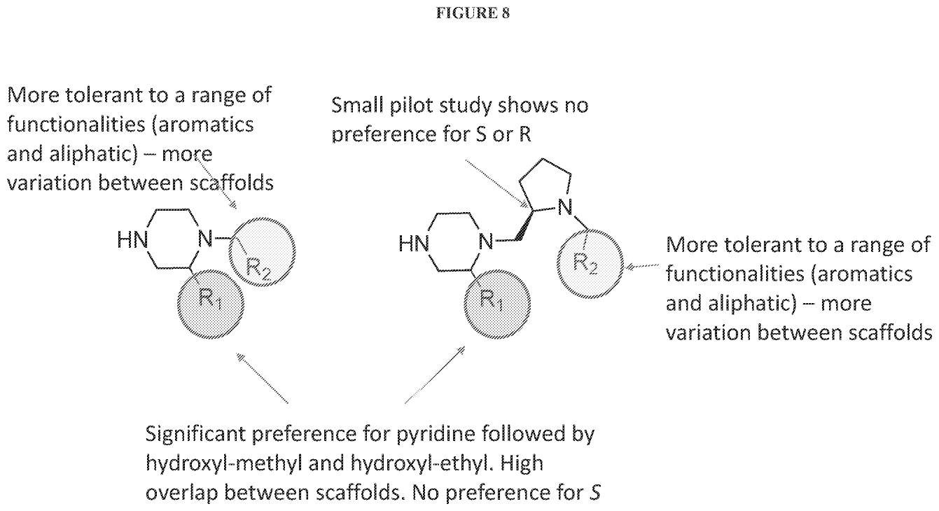

For the identification of CDR1 expression inhibitors the screening of the TPML collection began with a screen ranking 79 scaffolds. The use of the scaffold ranking screen allows for the rapid assessment of the different scaffolds in the entire collection, an approach which has been validated previously. The scaffold ranking screen included 79 different scaffolds covering over 30 million compounds. Each sample in the scaffold ranking screen contains only mixtures of compounds with the shared core scaffold. All compounds in a specific mixture are represented in approximately equal molar concentrations. The inventors performed the screen using the C. albicans azole-resistant, clinical isolate 5674, which was modified to carry a CDR1 allele tagged with GFP-encoding sequence. Modulation of Cdr1p-GFP levels in response to scaffold mixtures was assessed relative to basal levels (Response Ratio), from which scaffolds were ranked. Thus, a mixture that elicits a Response Ratio of 0.1, indicating a 90% reduction in Cdr1-GFP levels, is a more potent inhibitor of CDR1 expression than a scaffold eliciting a Response Ratio of 0.6. The inventors used tacrolimus at 10 μM concentration as positive control in all screening assays. This compound consistently elicited a Response Ratio of 0.8, indicating that the response to tacrolimus, a standard, was consistent, day to day, assay to assay. The 79 scaffold samples were screened at 50 ug/mL and 25 ug/mL, of which 18 samples were identified as potential scaffold hits for follow-up dose response (see FIG. 3, Table 2). Of the 18 scaffold samples selected for dose response studies, 8 of them had a piperazine functionality as a key feature in the core scaffold (FIG. 3, Table 2 —“Name” blue highlighted) and 6 of them could be classified as polyamines (FIG. 3, Table 2—“Name” green highlighted), suggesting the potential importance of these scaffold features. The activity reported in Table 2 is the ratio of response, the lower the value the greater the activity. Piperazines and polyamines have both been reported to have antifungal activity including against different species of Candida but to date it is not believed that their activity has been associated with CDR1 expression as the target so this could suggest a possible novel mechanism of action for this class of compounds. For some of these reported studies, other groups tried to optimize fluconazole by utilizing a piperazine scaffold with azole functionalities. Note, none of the piperazine libraries contain azoles as functional groups. There is at least one study showing that a compound containing a piperazine moiety has synergistic activity with fluconazole against C. albicans, though its mechanism of action was not described. The majority of the polyamines reported to date are derived from natural product extracts; our ability to synthesize unique polyamines from peptide-like starting points allows us to screen additional chemical space in this class. Based on this data, the inventors prioritized scaffolds 1324, 2220, 2222, and 2227 (FIG. 3, Table 2 and FIG. 4) to advance to the positional scanning screen. Scaffold 1324 occupies a unique chemical space (diketopiperazine linked to a dihydroimidazolyl) compared to the other hit scaffolds (piperazines and polyamines) and was among the scaffolds producing the best ratio responses at the different doses. While the dihydroimidazolyl moiety is not technically an azole due to the lack of aromaticity, it will be important to understand how this motif will behave in the fluconazole resistant lines. Libraries 2220 and 2222 were selected to represent the piperazine class based on their response ratios. Additionally, the individual compounds that make up each of these two libraries all have the same R group functionalities (FIG. 7); therefore, the data from the positional scan screening of these two libraries will enable a further evaluation of the SAR across these two different piperazine scaffolds. If libraries 2220 or 2222 did not lead to the identification of active individual compounds, then the libraries for 1664 and 1666 were to be screened to further assess the piperazine scaffolds. Finally, the 2227 scaffold was advanced to positional scanning. It was among the better polyamine scaffolds by response ratio. Additionally, the individual compounds in this scaffold have lower molecular weights on average than those of any of the other polyamine scaffolds, potentially allowing for more flexibility in hit optimization. Likewise, if no hit individual compounds were identified from this scaffold, libraries 1665 and 2161 would be screened to further evaluate the polyamine scaffolds. Of note, 2227 has two primary amines on the scaffold, whereas 1665 has none and 2161 has one, which points to potential opportunities for introducing or removing primary amines as needed in the hit optimization phase.

### Positional Scan Screening and Individual Compounds

Positional scanning libraries have been utilized by groups for over two decades now, with hundreds of published papers describing their design. In brief, the libraries are systematically arranged such that all the compounds for a given scaffold are formatted into mixtures based on common R group functionalities, which allows for their deconvolution and the generation of SAR data across all the compounds in the library. Therefore, if a scaffold has 30 different R1 functionalities, 30 different R2 functionalities, and 30 different R3 functionalities, it follows that the positional scanning library will contain 30 different R1 samples, themselves each containing 900 compounds (30 R2×30 R3=900). Similarly, R2 will have 30 samples, as will R3. The screen requires 90 sample wells. Of note: because the samples are organized to contain only structural analogues, if a particular individual compound is active, then the mixture sample containing that compound may possess many more structural analogues that are also active. Thus, the effective concentration of active compounds in a given mixture is, in general, significantly higher than it would be if only one compound in the mixture was active.

### Library 1324

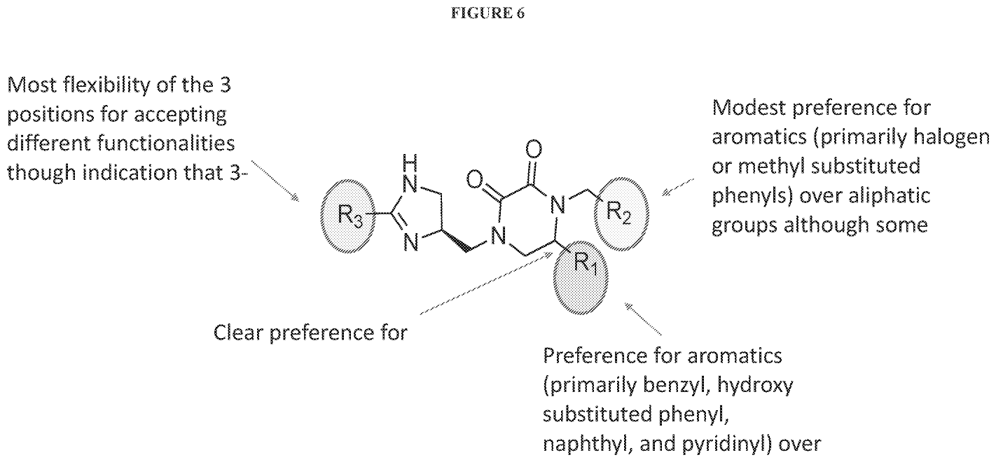

Positional scanning library 1324 contains 56,610 individual dihydroimidazolyl-methyl-diketopiperazines comprised from combining 34 R1 functionalities, 37 R2 functionalities, and 45 R3 functionalities (34 R1×37 R2×45 R3=56,610 in 34+37+45=116 sample wells) (FIG. 5), none of which had any azole functionalities. The 56,610 compounds are systematically synthesized into 116 mixture samples. The 116 samples were tested in the primary assay, with selected samples tested further in a dose response assay. Testing the 116 samples provides SAR data for the entire set of 56,610 compounds (FIGS. 5 and 6). The R1 position shows clear preference for the S configuration over the R configuration, although racemic mixtures are also suggested to be active and may be more effective for commercialization given that there would be less of a need for a stereospecific synthesis. Additionally, there is a preference seen for aromatic functional groups (primarily benzyl, hydroxy substituted phenyl, naphthyl, and pyridinyl) over aliphatic ones in the R1 position. The R2 functionality similarly shows a modest preference for aromatics (this time primarily halogen or methyl substituted phenyls) over aliphatic functionality, although there is some tolerance for cyclical or bulky aliphatic groups. The R3 position appears to allow for the most diversity, although the data indicates the 3-bromophenyl functional group may be the most optimal of the tested functionalities.

### Individual Compounds Based on 1324

The data was utilized to synthesize and test a set of individual dihydroimidazolyl-methyl-diketopiperazines in order to confirm the SAR and identify initial individual hit compounds from this class. In total, 164 individual dihydroimidazolyl-methyl-diketopiperazines were sent from TPI-FIU to UNM for testing in the primary assay. From the dose response studies with these compounds, 4 compounds from this class were advanced to secondary assay screening.

### Library 2220 and 2222

Positional scanning library 2220 and 2222 contains 2,340 individual piperazines and pyrrolidine piperazines, respectively, comprised from combining 39 R1 functionalities and 60 R2 functionalities (39 R1×60 R2=2,340) (FIG. 7), none of which had any azole functionalities. The 2,340 compounds were systematically synthesized into 99 mixture samples. The 99 samples were tested in the primary assay and selected samples were further tested in a dose response assay. Testing the 99 samples provided SAR data for the entire set of 2.340 compounds (FIGS. 7 and 8). The combined data indicated that there was no clear preference for S or R configuration at the R1 position for either scaffold. There is also a similar activity profile for the R1 position across the scaffolds as this is the common feature between the scaffolds. At the R1 position, there is a significant preference for a pyridine group, followed by hydroxyl-methyl and hydroxy-ethyl. There is more variation between scaffolds for the favored functionalities in the R2 position.

### Individual Compounds Based on 2220 and 2222

The data was utilized to synthesize and test sets of piperazine and pyrrolidine piperazines in order to confirm the SAR and identify initial individual hit compounds from this class. Additionally, a set of individual compounds was made to confirm the importance of stereochemistry at the pyrrolidine position of the 2222 scaffold. Based on the data, there appears to be no preference for either the S or R configuration (FIG. 8). In total, 119 individual piperazines and 119 individual pyrrolidine piperazines were tested. From the dose response studies with these compounds, 8 compounds from this class were advanced to secondary assay screening.

### Library 2227

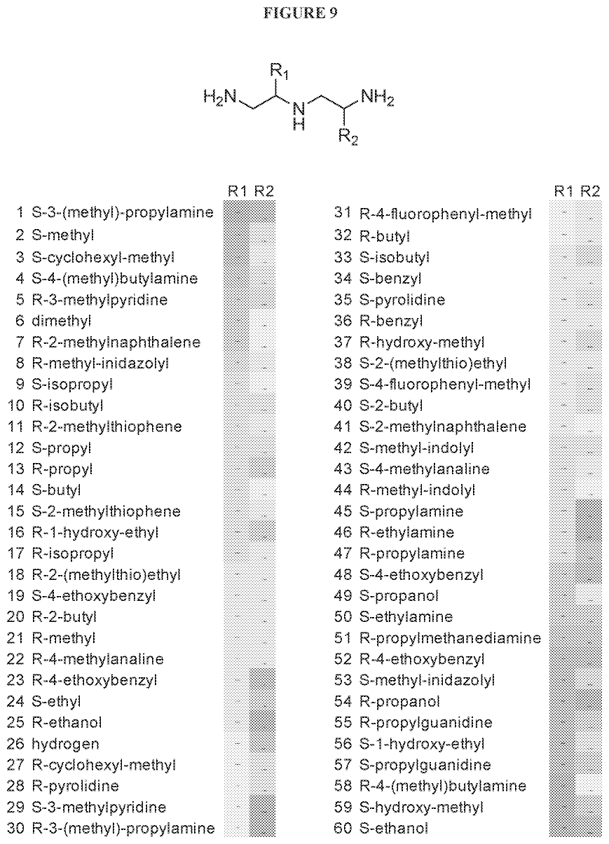

Positional scanning library 2227 contains 3,600 individual polyamines comprised from combining 60 R1 functionalities and 60 R2 functionalities (60 R1×60 R2=3,600) (FIG. 9), none of which had any azole functionalities. The 3,600 compounds were systematically synthesized into 120 mixture samples. The 120 samples were tested in the primary assay and selected samples were further tested in a dose response assay. Testing the 124 samples provides SAR data for the entire set of 3,600 compounds (FIG. 9). There was a trend with overlap of similar functionalities working in the R1 and R2 positions, with an even more striking trend in which some functionalities did not work in either position. The overlap in similar functionalities potentially leads to two classes of compounds: 1) symmetrical compounds that build off the symmetrical nature of the core scaffold and have similar functionalities in the R1 and R2; or 2) compounds that require diverse functionalities at the R1 versus R2 position (i.e., positively charged R1 with aliphatic ring in R2). The individual compounds tested (as described below) identified both classes as hits.

### Individual Compounds Based on 2227

The data was utilized to synthesize and test sets of polyamines in order to confirm the SAR from the library and identify initial individual hit compounds from this class. Also included were some individual polyamines to confirm the flexibility of expanding the chemical space of the polyamine scaffold to include the other polyamine scaffolds identified in the scaffold ranking library (FIG. 3. Table 2). In total, 164 individual polyamines were tested in the primary assay. From the dose response studies with these compounds, 4 compounds from this class were advanced to secondary assay screening. The SAR data from the 120 mixture samples combined with the individual compounds inform hit compound optimization.

### Secondary Screens

In this group of experiments, lead compounds were tested for (i) target specificity, and (ii) restoration of susceptibility and/or synergistic activity with fluconazole against azole-resistant strains. In these experiments, the inventors negotiated the following milestones. Based on estimates directly obtained from the preliminary data, the inventors identified active compounds that met the following criteria:

### (i) Target-Specific Inhibition of Cdr1p Expression (Non-Specific Anti-Fungal Activity at 5 Fold Selectivity).

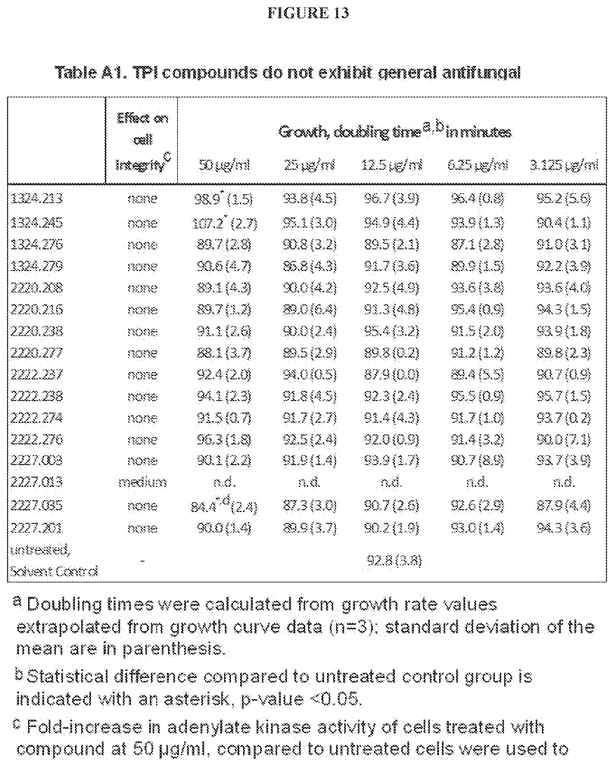

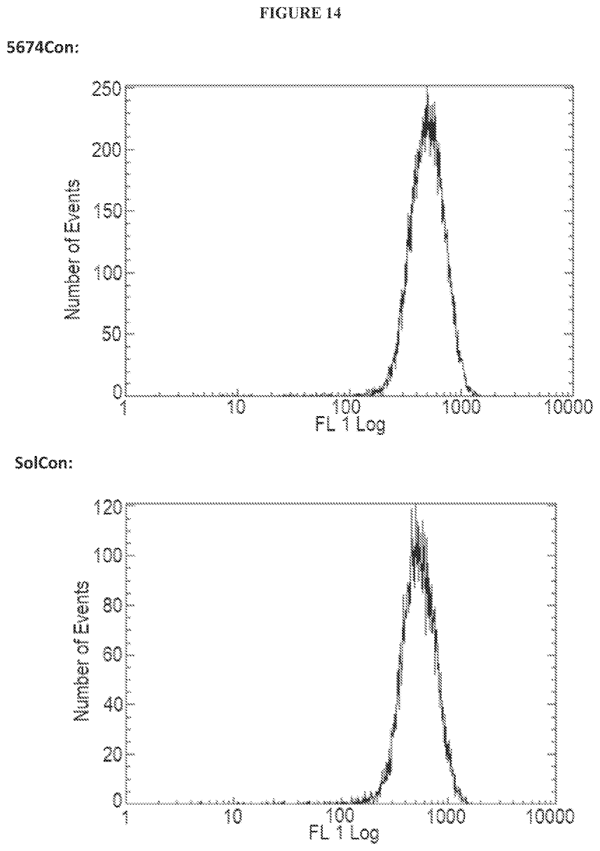

This screen identifies compounds that directly downregulate expression of the Cdr1p protein at the plasma membrane, as opposed to previously reported campaigns to find compounds that inhibit Cdr1p pump function. The assay readout, GFP fluorescence levels as detected by a flow cytometer, is directly related to the levels of Cdr1p of the cell. Thus, flow cytometry data from each screen provides direct evidence that Cdr1p is modulated by hit compounds. Subsequent assays served to eliminate false-positive hits and compounds that affect Cdr1p expression indirectly such as those compounds that have general antifungal activity. False-positive hits were identified in follow-up dose-response assays where the effect of serial dilutions of each compound on Cdr1p-GFP levels was assessed. Compounds that did not elicit a dose-response were likely false-positive hits and were eliminated from the workflow. In addition to the output from the dose-response assays, we included an experimental step designed to identify compounds with fungicidal potential. Specifically, the inventors used the assay supernatant to interrogate for the presence of adenylate kinase (AK), an enzyme that is released from the cells when the cell wall or plasma membrane integrity is compromised. Low-dose caspofungin (50 ng/ml), an echinocandin known for its antifungal property due to disruption of the cell wall, was used as a positive control. With the exception of compound 2227.013, all compounds that were tested (at 5× the concentration that results in 50% reduction in Cdr1p-GFP levels, EC₅₀) had similar AK levels to untreated control, suggesting that the compounds did not affect plasma membrane integrity (data not shown). Similar results were obtained when the inventors repeated the assay using Yeast Peptone Dextrose, as opposed to Complete Synthetic Medium, as the growth medium (see experiments below and FIG. 13, Table A1). It should be noted that potential fungicidal properties of 2227.013 at this high dose is considered mild compared to caspofungin (AK fold-increase of 27).

Next assessed was the potential impact of the hit compounds on growth using standard microtiter plate liquid growth assays and varying concentrations of each compound (up to 5 times the 10 μM EC₅₀ cut-off). The optical density, measured at 600 nm (OD₆₀₀ₙₘ), of each culture was followed every 15 minutes for a period of 30 hours. The growth rate at the exponential phase was extrapolated from plate reader data (OD₆₀₀ₙₘ vs Time) using the program GrowthRates, from which doubling times were calculated. Correlation coefficients for each condition were greater than 0.999 in all cases, supporting confidence and reliability of the growth rate values derived. The average growth rate from replicate cultures was compared to the average growth rate of replicate control cultures in which no compound (only solvent) was added, using the unpaired t-test with Welch's correction. All compounds, except 1324.245, 1324.213, and 2227.035, did not affect growth of C. albicans 5674 at all concentrations tested (Supplemental experiments, below and FIG. 13, Table A1). At the highest concentration tested, which is ≥5-fold higher than the calculated EC₅₀ values from the dose-response assays, the four compounds caused a slight increase (2227.035; p-value 0.0055) or decrease (1324.245 and 1324.213; p-values 0.0014 and 0.0018, respectively) in the growth rate compared to control. Importantly, no effect on growth was detected at concentrations within the 10 μM EC₅₀ cut-off activity criteria. Taken together, the inhibitory activity observed in each of the candidate compounds, which are able to elicit a 50% reduction in Cdr1p at a dose of 10 μM or lower, is not the indirect result of fungistatic or fungicidal properties.

### (ii) Inhibition of Cdr1 Expression by RT-qPCR at EC50.

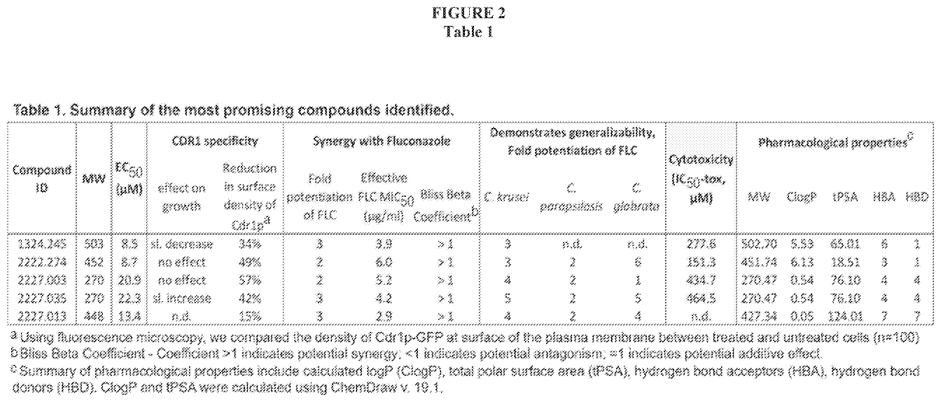

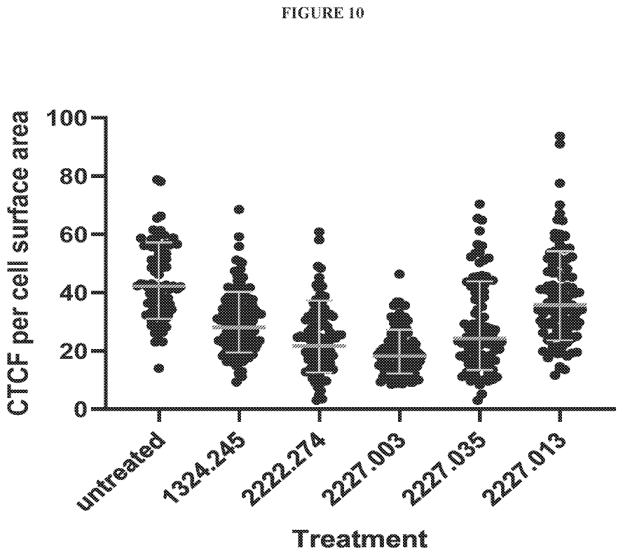

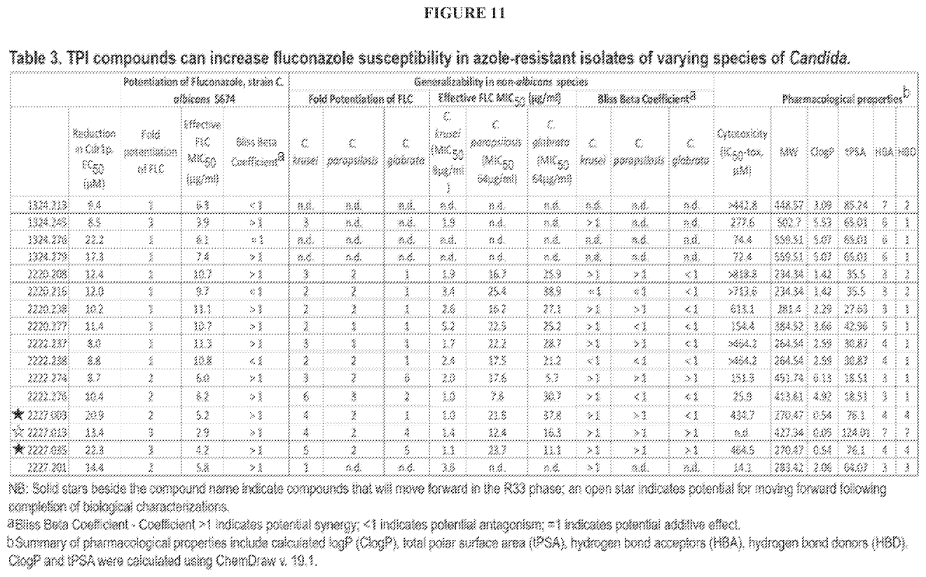

Next, the inventors sought to determine whether these compounds demonstrated target specificity, as determined by modulation of CDR1 gene expression. Thus, the inventors assayed gene expression of CDR1 in response to individual compounds using qPCR. To confirm down-regulation of Cdr1p protein levels at the plasma membrane (its functional cellular location), fluorescence images of cells grown in the presence of the top 5 compounds were collected using the same acquisition settings for all conditions. Only cells on the same focal plane, as supported by corresponding light micrographs, were processed and at least 100 cells from each condition was assessed. Plasma membrane fluorescence intensities (CTFC) were calculated using ImageJ, which were then expressed relative to the estimated surface area of the cell. This provides an estimate of the density of Cdr1p on the surface of the cell, whereby an azole-resistant strain is expected to have a higher surface density of Cdr1p than a susceptible strain. Compared to untreated cells, cells treated with compound had significantly reduced plasma membrane fluorescence intensities and significantly reduced Cdr1p surface density (FIG. 10), providing evidence that each compound effected a decrease in Cdr1p levels. It was noted that the fold-reduction in Cdr1p surface density effected by each compound did not directly correlate to the calculated fold-potentiation of fluconazole (FIG. 2, Table 1). This observation does not take away from the fact that these compounds reduce Cdr1p expression and provides support that the compound may have additional mechanisms of action that lead to synergy with fluconazole.

(iii) Synergistic Activity in Combination with Fluconazole (in the Vicinity of EC₅₀).

Overexpression of Cdr1p has been implicated in azole resistance in many fluconazole-resistant fungal pathogens. Based on previous studies and results from the preliminary data supporting this proposal, decreasing Cdr1p levels can increase azole susceptibility (i.e., decrease azole resistance) in clinical isolates that constitutively over-express Cdr1p. Thus, to further demonstrate specificity of the compounds identified in the screen, each compound's ability to increase fluconazole (FLC) susceptibility in the azole-resistant clinical isolate 5674 was assessed. The mechanism of azole-resistance in this strain is well defined, with significantly increased expression of CDR1 and CDR2 (genes encoding for ABC-type efflux pump), but not the MFS-type efflux pump MDR1. The inventors used the CLSI microbroth dilution method of antifungal susceptibility testing in a checkerboard assay format, where serial 2-fold dilutions of FLC (from 64 μg/ml) were tested in combination with serial 2-fold dilutions of a candidate hit compound (from 50 μg/ml). Of the 16 single compounds tested, 10 potentiated FLC activity, resulting in effective FLC MIC₅₀ values of less than 8 μg/ml (FIG. 11, Table 3), the MIC₅₀ that characterizes most Candida species as azole-resistant. The data from three biological replicates was next used to assess synergy between each compound of interest and FLC. For these purposes, the inventors used the program SynScreen, which extrapolates non-linear fits of dose-response data to determine synergy using the Bliss independence and Loewe additivity analysis models. Data points found above the plotted three-dimensional surface, that represents theoretical points at which Bliss additivity occurs, indicating synergy. The Bliss Beta coefficient that the program calculates for all combinations further describes potential synergism (if >1), antagonism (if <1), or an additive effect (equal to 1). Of the 10 compounds that effected a FLC MIC₅₀ of <8 μg/ml, 8 resulted in BlissBeta coefficients that predict synergy (FIG. 11. Table 3) for which each of the primary scaffolds identified with activity are represented.

These exciting and promising results prompted the inventors to test a small subset of known azole-resistant strains, to evaluate the therapeutic generalizability of these compounds in clinically relevant non-albicans strains. This included C. krusei reference strain, ATCC®6258™ (MIC₅₀ 8 μg/ml), C. glabrata AR Bank #0314 (MIC₅₀ 64 μg/ml), and C. parapsilosis AR Bank #0337 (MIC₅₀ 64 μg/ml), the last two were chosen from the CDC FDA Drug Resistant Candida species panel on the basis of their high azole-resistance profile (MIC, fluconazole). For the purposes of reporting the effect of the test compound on FLC potentiation, the inventors excluded from analyses rows of combinations where TPIML compound alone affects yeast growth. C. krusei are intrinsically resistant to azoles and is predominantly due to a significant reduction of inhibition of the target, cytochrome P450 lanosterol 14α-demethylase, though the role of increased expression of the CaCdr1p homolog in resistance has also been described. Remarkably, 6 out of the 8 strongest candidate compounds against C. albicans 5674 could also synergistically increase susceptibility of C. krusei ATCC®6258™ to effect an MIC₅₀ of 2 μg/ml or lower (FIG. 11, Table 3). That this observation is the direct result of decreased pump levels and subsequent increased levels of intracellular fluconazole, versus synergistic increase in fluconazole's affinity for its target, remains to be elucidated. The role of increased expression of the Cdr1p homologs in C. glabrata azole resistance has been previously established, with a third ABC-type transporter (CgSnq2) playing a similar role. All three ABC-type transporters are under the transcriptional control of the zinc cluster transcription factor, CgPdr1p. The inventors have tested 5 of the 8 best potentiators identified against C. albicans, against C. glabrata AR Bank #0314. Three of these compounds synergistically increased FLC susceptibility by 4 to 6-fold, potentiating FLC to yield an MIC₅₀ between 6 and 16 μg/ml (FIG. 11, Table 3). Notably, these values fall within the susceptible dose-dependent range of fluconazole for C. glabrata MIC clinical breakpoints. Azole resistance in C. parapsilosis can also result from overexpression of its ABC-type transporter, CpCdr1p, but other mechanisms of azole resistance may come into play, similar to findings in C. albicans. Results show that the abilities of the TPIML compounds to potentiate fluconazole in C. parapsilosis AR Bank #0337 were modest compared to their effects on the other Candida species tested (FIG. 11, Table 3).

Synergy falls within the concentration at which the compound reduces Cdr1p fpr compounds 2227.003, 2227.013, and 2227.035. The concentrations at which the maximum potentiation of fluconazole is achieved by compounds 2227.003, 2227.013, and 2227.035 are 11.3 μM, 7.7 μM, and 11.3 μM, respectively. Compounds 2227.003 and 2227.035 were synthesized based on SAR information derived from the screening of Positional Scanning Libraries.

### (iv) Marginal Mammalian Cell Toxicity.

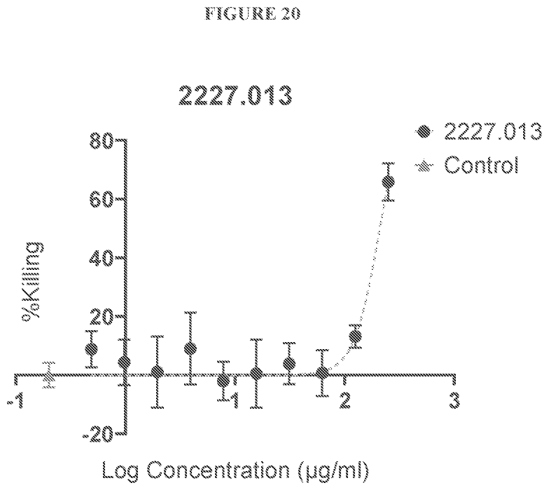

Safety for use in humans is an important aspect of any drug discovery campaign. As the hepatic system represents one of the most commonly susceptible tissue types that affect in vivo toxicity, the inventors used the human HepG2 cell line, Hep G2 [HEPG2] (ATCC® HB8065™) to assess potential mammalian cytotoxicity properties of each compound of interest. Confluent, adhered HepG2 cells were incubated with ten 2-fold dilutions of compound (starting at 250 μg/ml) for 24 hours. Cell viability was then assessed using the CyQUANT™ XTT Cell Viability Assay (Invitrogen) following manufacturer instructions. Data was expressed as the proportion of respired cells and the concentration at which each compound resulted in 50% death (IC₅₀₋ₜₒₓ) was calculated. The assay was performed in triplicate, with four replicates per compound of interest, per assay. The inventors were interested in compounds that did not possess cytotoxic properties at concentrations 5 times that which result in 50% reduction of Cdr1p. This roughly equates to 50 to 120 μM. Of the 16 TPI compounds assessed, four exhibited significant toxicity against the hepatic cell line, with IC₅₀₋ₜₒₓ, values of less than 100 μM (FIG. 11. Table 3). Two of these compounds, 2222.276 and 2227.201, increased fluconazole potentiation in more than one species of Candida but had no discernable effect on either growth or plasma membrane integrity of C. albicans strain 5674 (First Supplemental Experiment). However, the hepatotoxicity drastically reduces the therapeutic window for safety. Five compounds show a potential therapeutic window (FIG. 20.

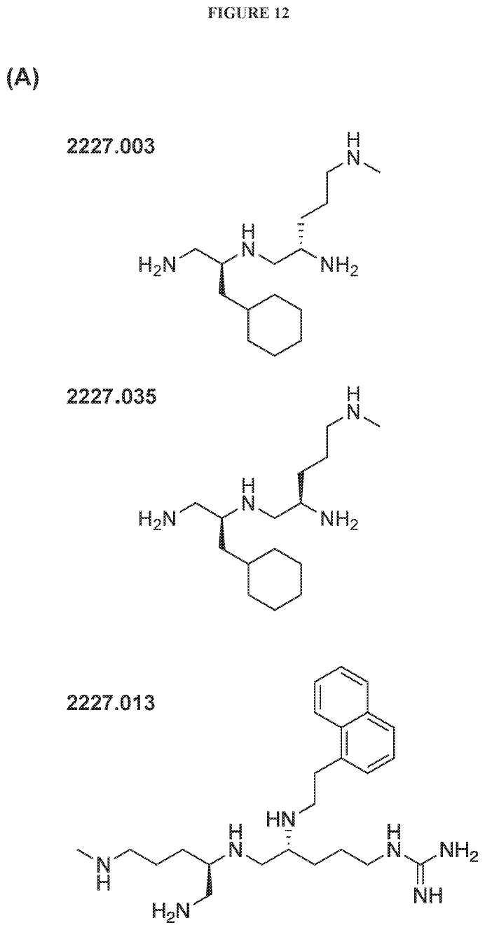

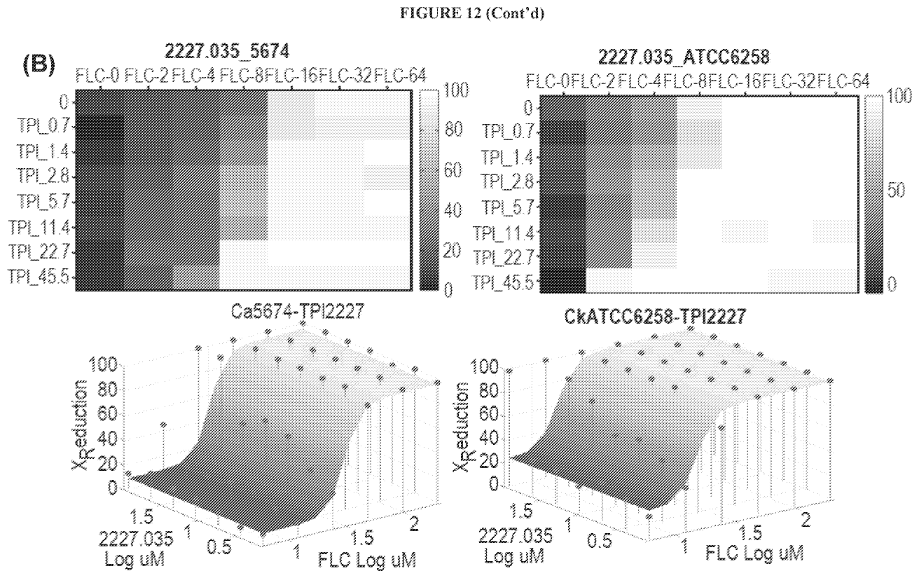

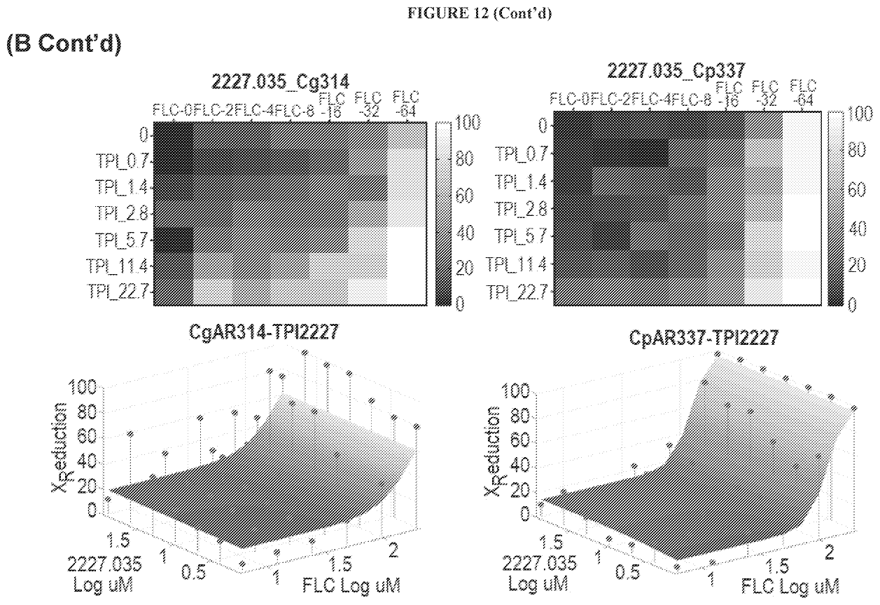

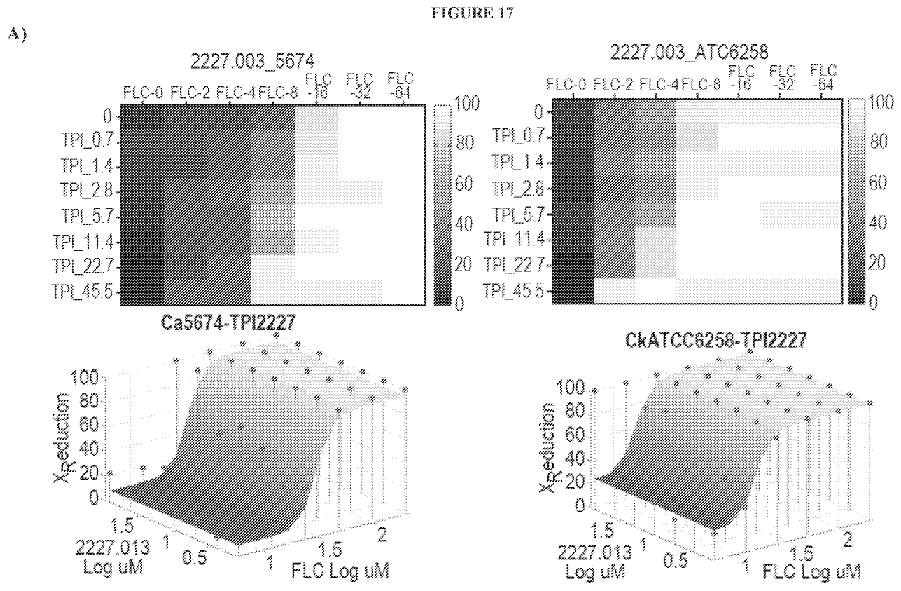

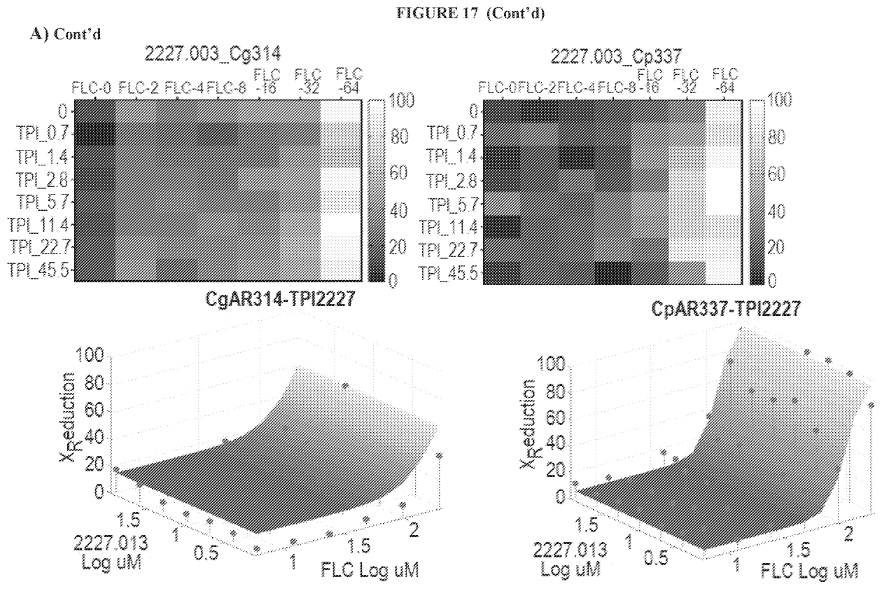

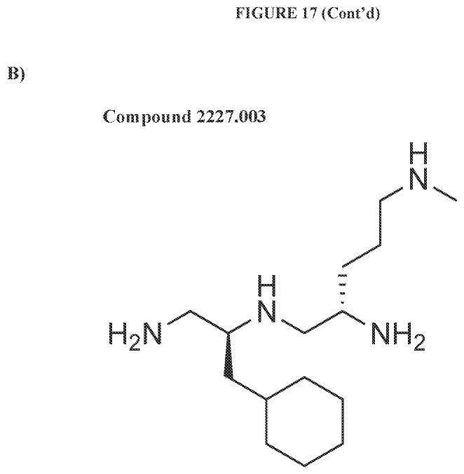

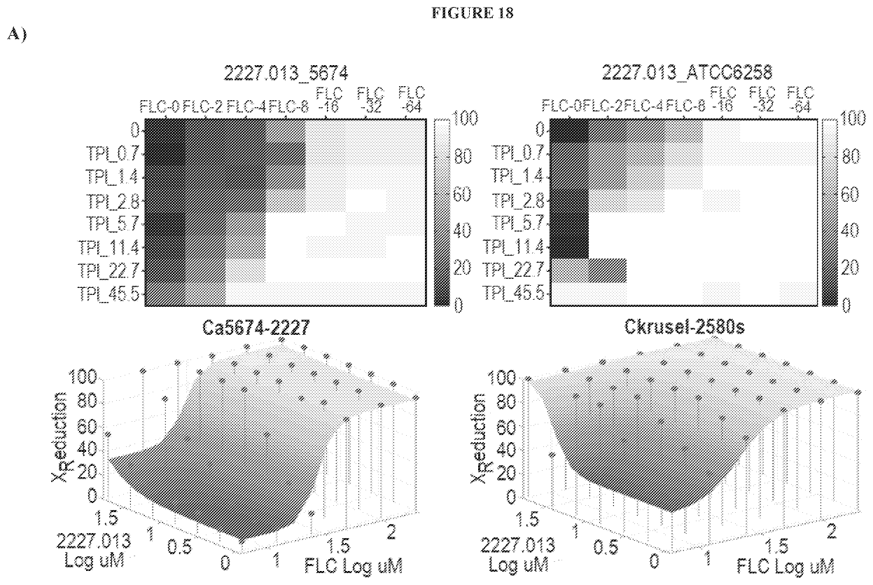

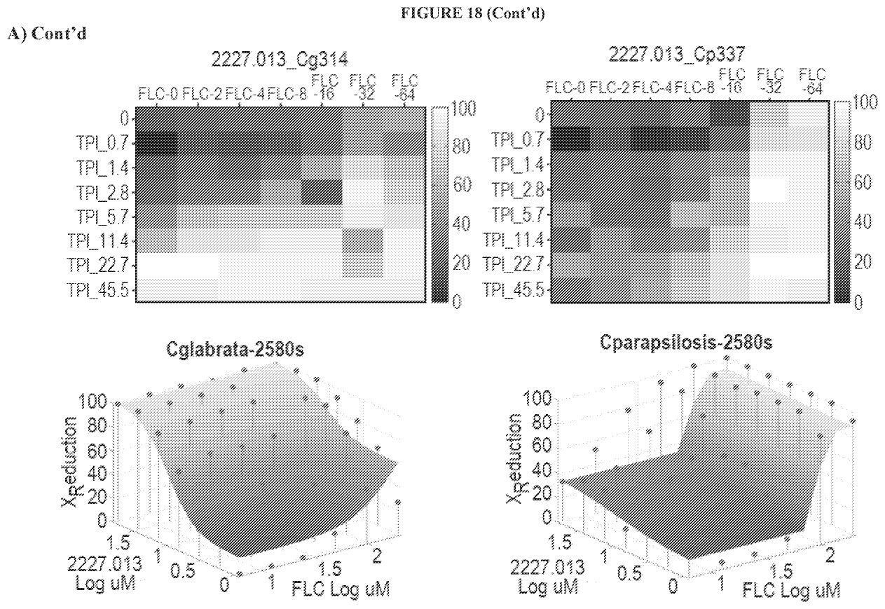

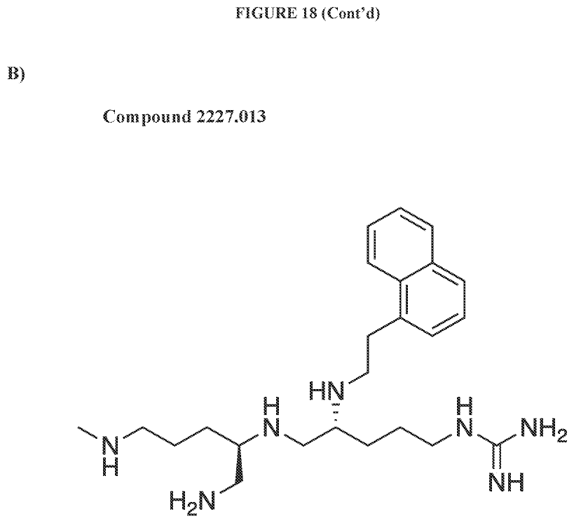

In summary, experiments (including supplemental experiments, described herein below) have identified 3 compounds (2227.003, 2227.013 and 2227.035), among others, that represent particularly useful compounds for use in treating fungal infections to address fungal resistance to azole antifungal agents. These compounds do not appear to have general antifungal properties and, along with their ability to inhibit Cdr1p expression, potentiate a synergistic reduction in fluconazole resistance of appropriate magnitude in clinically relevant Candida species (FIG. 11, Table 3). Compounds 2227.013, 2227.035, 1324.245 and 2222.274 show significant potential for use as agents to reduce fungal resistance to azole antifungal agents. The structure of these compounds and results from a representative compound, 2227.035, are shown in FIG. 12, FIG. 17 and FIG. 18. Other compounds require optimization of potentiation (1324.245 and 2222.274) (Supplemental Experiment).

### Supplemental Experiments

### Inhibition of Cdr1 Expression by RT-qPCR.

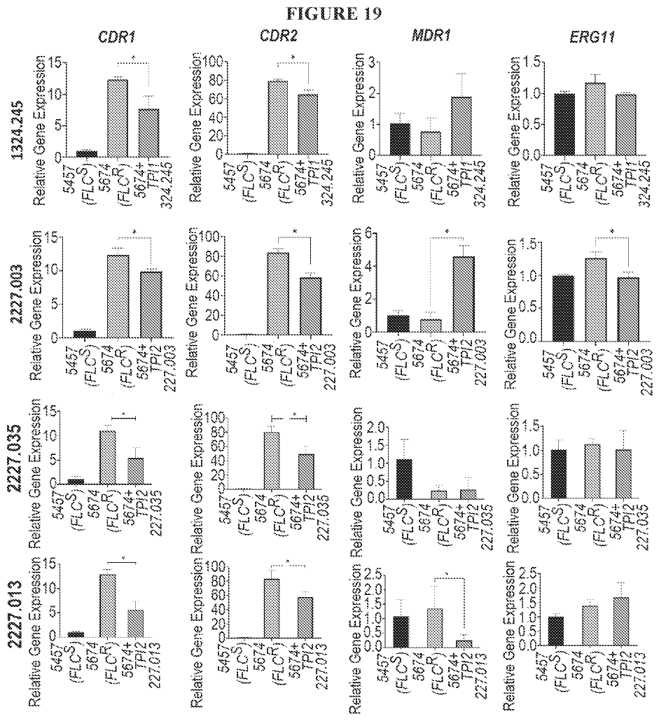

In the presence of TPI compounds, the levels of Cdr1p at the plasma membrane of the azole-resistant clinical isolate 5674 were significantly decreased compared to those in untreated cells. The reduction of the protein at the site at which they function to remove intracellular fluconazole, helps to restore azole sensitivity to azole-resistant yeast strains. RT-qPCR primers were designed to amplify regions of the open reading frame within 1.5 kb of the predicted transcript stop codon. This design avoids the impact of potential inefficiencies of cDNA amplification reactions that may lead to incomplete synthesis of cDNA and aberrant RT-qPCR results using primers that anneal to the 5′ region of the transcript. Three replicate RT-qPCR reactions were run for each of the total RNA samples extracted from three biological replicates of cells grown in the presence and absence of TPI compounds. The matched fluconazole-sensitive clinical isolate 5457 was included in the analysis as the normalizing reference. Primer efficiencies, calculated from calibration curves using pooled cDNA samples from all conditions tested, were verified to be within 90 and 110%. Data was acquired for each target transcript (CDR1, CDR2, ERG11, and MDR1) and reference genes (ACT1, PMA1, RIP, and RPP2B) in 384-well format. Performance of reference genes within each RT-qPCR reaction plate was assessed using NormFinder and the best two reference genes were used in the Vandesompele method for calculating relative gene expression, normalized to expression levels in the matched fluconazole-sensitive strain 5457. Reduction of target transcript in strain 5674 grown in the presence of TP1 compound was compared to the same strain grown in the absence of TPI compound using the unpaired t-test. Using the newly designed primers the inventors were able to verify that the compounds decreased expression of both ABC-type efflux pumps, CDR1 and CDR2, by 20-57% and 18-39%, respectively (FIG. 19). Despite effecting an apparent increase in expression of MDR1, which encodes an MFS-type efflux pump whose overexpression can impart fluconazole resistance, TP12227.003 potentiated fluconazole against Candida species. It remains to be determined if this increase in transcript correlates to increased levels of the protein itself. Interestingly, only compound 2227.013 additionally downregulated MDR1 by about 80% of wildtype-level expression. This was a serendipitous surprise as MDR1 is under transcriptional control of Mrr1p, independent of the Tac1p-regulated ABC-type efflux pumps. This could explain the increased fluconazole-potentiation activity of 2227.013 compared to all other compounds, and thus this compound may have broader application against azole-resistant clinical isolates bearing more than one mechanism of resistance. The inventors checked the expression of ERG/I which encodes the target of fluconazole and found that expression was unchanged in the presence of our compounds. Altogether, these results support the idea that the organism is unable to counter the effect of most of our compounds by invoking other known mechanisms of antifungal resistance (eg, increased expression of either the azole drug target or other efflux pumps), indicating that development of resistance to the compounds is unlikely.

### Mammalian Cell Toxicity.

Safety for use in humans is an important aspect of any drug discovery campaign. As the hepatic system represents one of the most commonly susceptible tissue types that affect in vivo toxicity, the inventors used the human HepG2 cell line, Hep G2 [HEPG2] (ATCC® HB8065™) to assess potential mammalian cytotoxicity properties of TPI2227.013. Confluent, adhered HepG2 cells were incubated with ten 2-fold dilutions of compound (starting at 250 μg/ml) for 24 hours. Cell viability was then assessed using the CyQUANT™ XTT Cell Viability Assay (Invitrogen) following manufacturer instructions. Data was then expressed as the proportion of respired cells and the concentration at which each compound resulted in 50% death (IC₅₀₋ₜₒₓ) was calculated. The assay was performed with four replicates per compound of interest. We were interested in compounds that did not possess cytotoxic properties at concentrations 5 times that which result in 50% reduction of Cdr1p (EC₅₀). For compound TPI2227.013 this equates to 67 μM. The inventors have shown that only the highest dose tested exhibited cytotoxicity (FIG. 20) and that the estimate for I₅₀₋ₜₒₓ is 209.1 μg/ml. This IC₅₀₋ₜₒₓ is at least 35 times higher than the concentration needed to decrease Cdr1p expression by 50% (EC₅₀), thus representing a wide therapeutic window for compound 2227.013.

### Growth in the Presence of TPI2227.013.

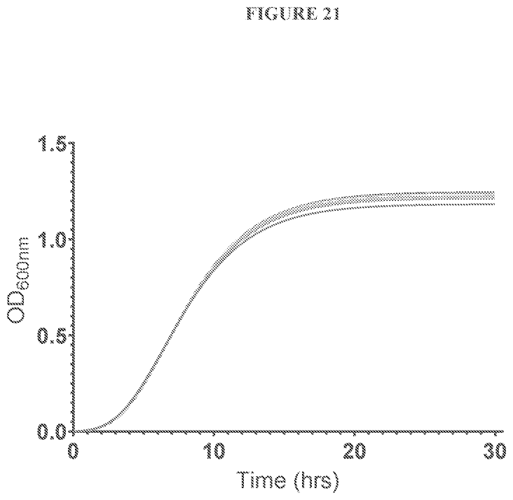

The inventors assessed the potential impact of compound TPI2227.013 on growth using standard microtiter plate liquid growth assays and varying concentrations of TP12227.013 (up to 50 μg/ml, or ˜8 times the EC₅₀). The optical density, measured at 600 nm (OD₆₀₀ₙₘ), of each culture replicate was followed every 15 minutes for a period of 30 hours. The growth rate at the exponential phase was extrapolated from plate reader data (OD₆₀₀ₙₘ vs Time) using the program GrowthRates as previously described (see Hall, et al., Molecular biology and evolution, 2014. 31(1): p. 232-8), from which doubling times were calculated. Correlation coefficients for each condition were greater than 0.999 in all cases, supporting confidence and reliability of the growth rate values derived. The average growth rate from replicate cultures grown in the presence of TP12227.013 was compared to the average growth rate of control cultures in which no compound (only solvent) was added, using the unpaired t-test with Welch's correction. At all doses tested (3.125 to 50 μg/ml), TP12227.013 did not affect the growth rate of C. albicans (p<0.0001). Thus, the decrease in Cdr1p-GFP observed is not due to general antifungal properties of TPI2227.013. See FIG. 21.

### CONCLUSION

In conclusion, the inventors have discovered small molecules that can target the pathways that give rise to the expression of CDR1 (Cdr1p) and in embodiments CDR2 (Cdr2p), both ABC-type plasma membrane transporters, which are major contributors to antifungal therapy resistance. These compounds can be used alone or preferably in conjunction with azole antifungal agents to provide particularly effective antifungal therapy, in many instances the action is synergistic against the fungal infection (through inhibition of growth or for its fungicidal activity).

1. A pharmaceutical composition comprising an effective amount of at least one CDR1 inhibitor compound in combination with an azole antifungal agent and a pharmaceutically acceptable carrier, additive or excipient.

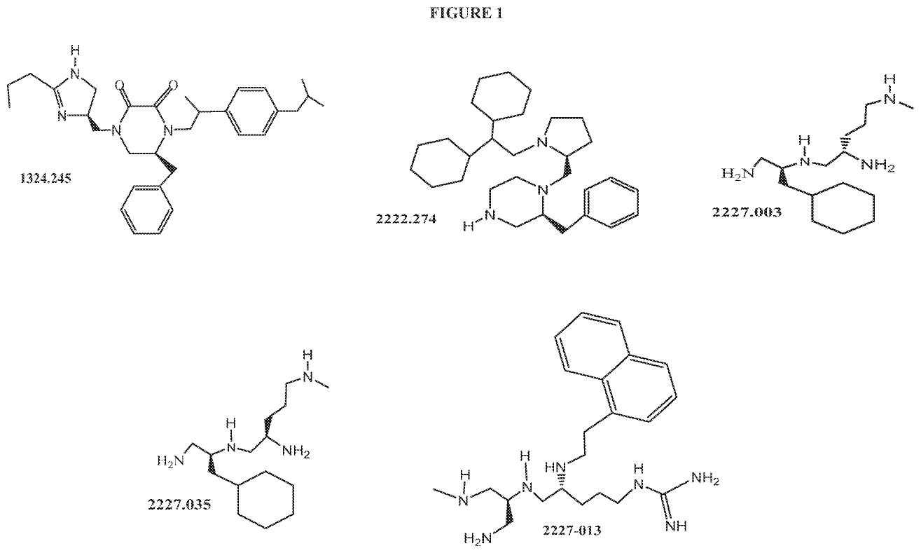

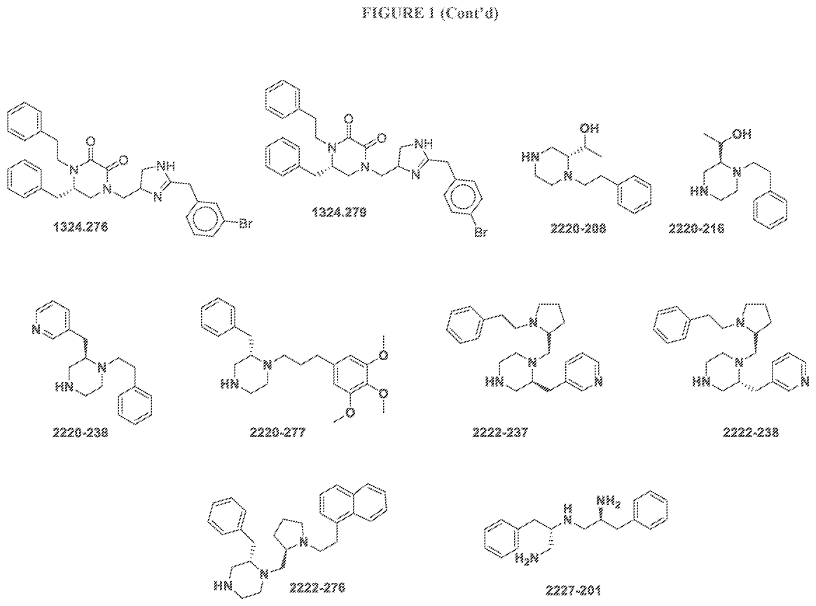

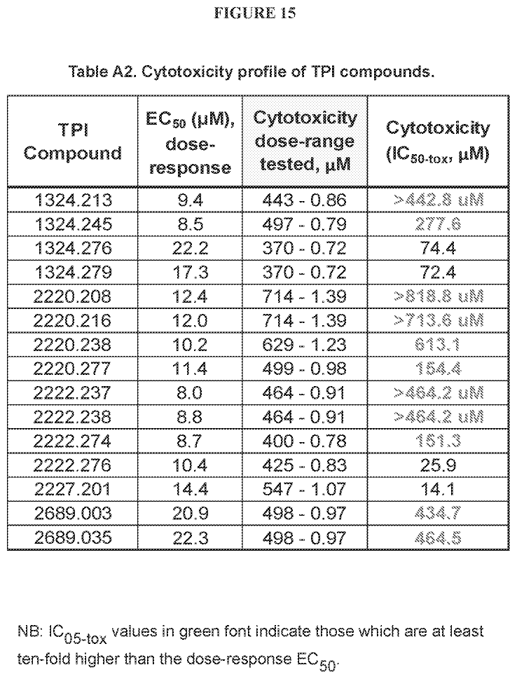

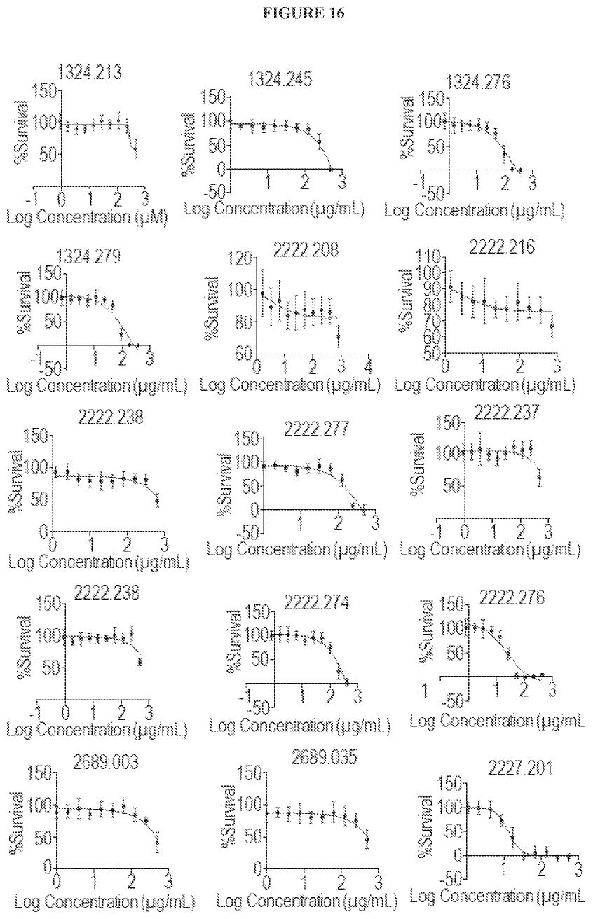

2. The composition according to claim 1 wherein said inhibitor compound is a compound which is set forth in FIG. 1, FIG. 2, FIG. 3, FIG. 4, FIG. 5, FIG. 6, FIG. 7, FIG. 8, FIG. 9, FIG. 11, FIG. 12, FIG. 13, FIG. 15, FIG. 16, FIG. 17, FIG. 18, FIG. 19 hereof, or a pharmaceutically acceptable salt thereof or mixture thereof.

3. The composition according to claim 1 wherein said inhibitor compound is a compound as set forth in FIG. 5, FIG. 7 or FIG. 9 hereof.

4. The composition according to claim 1 wherein said compound is 1324.245, 2222.274, 2227.003, 2227.035, 2227.013, a mixture thereof or a pharmaceutically acceptable salt thereof.

5. The composition according to claim 1 wherein said inhibitor compound is 1324.276, 1324.279, 2220-208, 2220-216, 2220-238, 2222-237, 2222-238, 2222-276, 2227-201 or a pharmaceutically acceptable salt, diastereomer, enantiomer, solvate, polymorph or a mixture thereof.

6. The composition according to claim 1 wherein said inhibitor compound is a compound according to the chemical structure 1324: 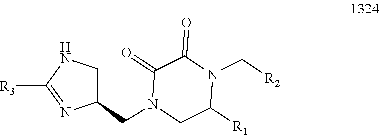
Where R₁ is H, C₁-C₁₂ (often C₁-C₆ or C₁-C₃) alkyl (optionally substituted with 1-3 halogen groups F, Cl, Br or I groups or 1-2 hydroxyl groups), naphthyl, 2-naphthylmethyl, 4-hydroxybenzyl, pyridin-3-yl-methyl, phenyl (optionally substituted with from 1-3 halogen groups or 1-2 hydroxyl groups), benzyl (optionally substituted with from 1-3 halogen groups and/or 1-2 hydroxyl groups) or pyridine-3-yl-methyl (often R₁ groups include S-2-naphthylmethyl, R-2-naphthylmethyl, S-4-hydroxybenzyl, S-pyridin-3-yl-methyl, S-benzyl, gamma-butyl, S-isobutyl, S-propyl, S-2-butyl, R-methyl, R-propyl, R-phenyl, S-tert-butyl, S-isopropyl, S-butyl, R-benzyl, R-2-butyl, R-pyridin-3-yl-methyl, S-4-fluoro-benzyl, R-isopropyl, S-cyclohexyl, R-butyl, S-methyl, R-isobutyl or R-tert-butyl);
R₂ is H, C₁-C₁₂ (often C₁-C₆ or C₁-C₃) alkyl (optionally substituted with 1-3 halogen groups F, Cl, Br or I groups or 1-2 hydroxyl groups) or —(CH₂)ₙphenyl (optionally substituted with 1-3 halogen groups, 1-2 hydroxyl groups, 1-3 —O—C₁-C₃) alkyl groups, and/or 1-3 optionally substituted C₁-C₄ alkyl groups which are optionally substituted with 1-3 halogen or 1-2 hydroxyl groups) (R₂ groups often include 2-(3-bromo-phenyl)-ethyl, m-tolylethyl, 2-(3-fluoro-phenyl)-ethyl, isobutyl, 2-Bicyclo[2.2.1]hept-2-yl-ethyl, cyclohexyl-butyl, cyclobutyl-methyl, n-butyl, ethyl, phenylpropyl,4-methyl-1-cyclohexylmethyl, cycloheptyl-methyl, 2-phenylbutyl, 2-(3,5-bis-trifluoromethyl-phenyl)-ethyl, 2-(4-Isobutyl-phenyl)-propyl, 3-cyclopentyl-propyl, cyclohexyl-methyl, (+/−)-2-Methylbutyl, cyclopentyl-methyl, heptyl, 2-(3-methoxy-phenyl)-ethyl, 4-phenylbutyl, 3-(3,4-dimethoxy-phenyl)-propyl, 3-methylbutyl or phenethyl);
R₃ is H, C₁-C₂₀, often C₁-C₁₂ (often C₁-C₆ or C₁-C₃) alkyl (optionally substituted with 1-3 halogen groups F, Cl, Br or I groups or 1-2 hydroxyl groups), C₂-C₆ alkene, often a vinyl group which is optionally substituted, —(CH₂)ₙphenyl (optionally substituted with 1-3 halogen groups, 1-2 hydroxyl groups, 1-3 —O—C₁-C₃) alkyl groups, and/or 1-3 optionally substituted C₁-C₄ alkyl or alkene groups which are optionally substituted with 1-3 halogen or 1-2 hydroxyl groups), —(CH₂)ₙ-biphenyl (optionally substituted with 1-3 halogen groups, 1-2 hydroxyl groups, 1-3 —O—C₁-C₃) alkyl groups, and/or 1-3 optionally substituted C₁-C₄ alkyl or alkene groups which are optionally substituted with 1-3 halogen or 1-2 hydroxyl groups) with the proviso that R1, R₂ and R₃, are not each simultaneously H, preferably no more than one of R1, R₂ and R₃ is H (R₃ groups often include 3-bromo-benzyl, 4-fluoro-phenyl, 4-methyl-cyclohexyl, 3-methyl-phenyl, tert-butyl, Bicyclo[2.2.1]hept-2-yl-methylcyclopentyl, ethyl, cyclobutyl, 4-methoxy-benzyl, 2-cyclohexyl-ethyl, cycloheptyl, propyl, biphenyl-4-yl, 2-(3,4-dimethoxy-phenyl)-ethyl, biphenyl-4-yl-methyl, 3,4-dimethoxy-benzyl, 4-bromo-benzyl, 1-ethyl-pentyl, 2-(2-trifluoromethyl-phenyl)-vinyl, 2-cyclopentyl-ethyl, hexyl, 3-fluoro-benzyl, benzyl or 3-phenyl-propyl; and n is 0-6, often 0-4, or a pharmaceutically acceptable salt, stereoisomer, diastereomer, enantiomer, solvate or polymorph thereof.

7. The composition according to claim 1 wherein said inhibitor compound is a compound according to the chemical structure:
a compound according to the chemical structure 2220 or 2222: 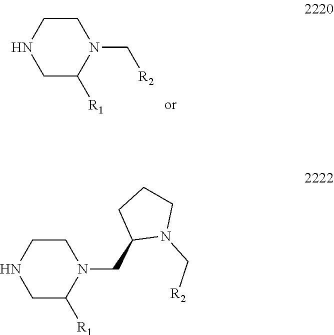
Where R₁ is H, C₁-C₁₂ (often C₁-C₆ or C₁-C₃) alkyl (optionally substituted with 1-3 halogen groups F, Cl, Br or I groups or 1-2 hydroxyl groups), naphthyl, optionally substituted with from 1-3 C₁-C₃ alkyl groups, pyridine, optionally substituted with 1 or 2 C₁-C₃ alkyl groups, —(CH₂)ₙphenyl (optionally substituted with from 1-3 halogen groups, 1-2 hydroxyl groups or 1-3 C₁-C₃ alkyl groups, which are optionally substituted with from 1-3 halogen, often fluoro), (often R₁ is R-3-methylpyridine, S-3-methylpyridine, R-hydroxy-methyl, S-1-hydroxy-ethyl, R-isobutyl, S-hydroxy-methyl, S-4-hydroxybenzyl, R-propyl, R-1-hydroxy-ethyl, S-propanol, S-4-fluorophenyl-methyl, R-methyl, S-methyl, R-butyl, S-isopropyl, R-4-fluorophenyl-methyl, S-propyl, R-2-butyl, R-cyclohexyl-methyl, R-isopropyl,
S-2-butyl, S-dimethyl, R-4-chlorophenyl-methyl or R-2-methylnaphthalene);
R₂ is C₁-C₁₂ (often C₁-C₆ or C₁-C₃) alkyl (optionally substituted with 1-3 halogen groups F, Cl, Br or I groups or 1-2 hydroxyl groups), a thiophenyl, furanyl or indolyl group each of which is optionally substituted with from 1-3 C₁-C₃ alkyl groups, —(CH₂)ₙphenyl (optionally substituted with from 1-3 halogen groups, 1-2 hydroxyl groups or 1-3 —O—C₁-C₃ alkyl or 1-3 C₁-C₃ alkyl groups, which are optionally substituted with from 1-3 halogen, often fluoro groups) (R₂ is often 2-thiophene-methyl, m-tolylethyl, 2-(3-methoxy-phenyl)-ethyl,
2-(3-trifluoromethyl-phenyl)-ethyl, 2-methylfuran, 2-(3-fluoro-phenyl)-ethyl, 2-methylbutyl,
m-xylene, 4-methyl-benzyl, heptyl, butyl, t-butylmethyl, 4-methoxy-benzyl, (1-phenyl-cyclopropyl)-methyl, p-tolylethyl, 4-ethyoxyphenylethyl, dicyclohexyl-ethyl, 3-indolylethyl
2-(4-methoxy-phenyl)-ethyl, cyclohexyl-ethyl, cyclohexyl-butyl, 3,4-dichlorophenethyl,
phenethyl, cyclohexyl-methyl or toluene); and n is 0-6, often 0-4, or a pharmaceutically acceptable salt, stereoisomer, diastereomer, enantiomer, solvate or polymorph thereof.

8. The composition according to claim 7 wherein said inhibitor compound is according to the chemical structure 2220: 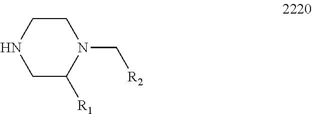
a pharmaceutically acceptable salt, diastereomer, enantiomer, solvate or polymorph thereof.

9. The composition according to claim 7 wherein said inhibitor compound is according to the chemical structure 2222: 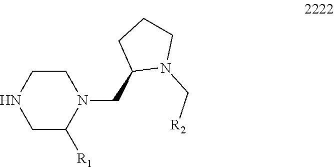
or
a pharmaceutically acceptable salt, diastereomer, enantiomer, solvate or polymorph thereof.

10. The composition according to claim 1 wherein said inhibitor compound is a compound according to the chemical structure 2227: 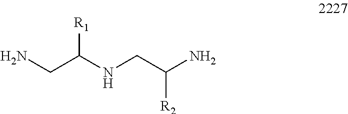
Where R₁ and R₂ are each independently H, C₁-C₁₂ (often C₁-C₆ or C₁-C₃) alkyl (optionally substituted with 1-3 halogen groups F, Cl, Br or I groups, 1-2 hydroxyl groups or an amine group which is optionally substituted with one or two methyl groups), naphthyl optionally substituted with 1-3 C₁-C₃ alkyl groups, pyridine, thiophene or imidazole, each of which is optionally substituted with 1 or 2 C₁-C₃ alkyl groups, C₂-C₆ thioether, naphthyl, optionally substituted with from 1-3 C₁-C₃ alkyl groups, —(CH₂)ₙphenyl (optionally substituted with from 1-3 halogen groups, 1-2 hydroxyl groups, 1-3 —O—C₁-C₃ alkyl groups or 1-3 C₁-C₃ alkyl groups, which are optionally substituted with from 1-3 halogen, often fluoro), analine, optionally substituted with from 1-3 C₁-C₃ alkyl groups, with the proviso that R₁ and R₂ are not both H (often R₁ and R₂ are each independently S-3-(methyl)-propylamine, S-methyl,
S-cyclohexyl-methyl, S-4-(methyl)butylamine, R-3-methylpyridine, dimethyl, R-2-methylnaphthalene, R-methyl-imidazolyl, S-isopropyl, R-isobutyl, R-2-methylthiophene, S-propyl, R-propyl, S-butyl, S-2-methylthiophene, R-1-hydroxy-ethyl, R-isopropyl, R-2-(methylthio)ethyl, S-4-ethoxybenzyl, R-2-butyl, R-methyl, R-4-methylanaline, R-4-ethoxybenzyl, S-ethyl or R-ethanol; and n is 0-6, often 0-4, or a pharmaceutically acceptable salt, stereoisomer, diastereomer, enantiomer, solvate or polymorph thereof.

11. The composition according to claim 1 wherein said azole antifungal agent is selected from the group consisting of voriconazole, itraconazole, posaconazole, ketoconazole, fluconazole, clotrimazole, isavuconazonium, miconozale, econazole, sulconazole, oxiconazole, cyproconazole, defnoconazole, etaconazole, fenbuconazole, flusilazole, hexaconazole, propiconazole, tebuconazole, triademenol, uniconazole, epoxiconazole or a pharmaceutically acceptable salt or mixture thereof.

12. The composition according to claim 1 wherein said azole antifungal agent is selected from the group consisting of voriconazole, itraconazole, posaconazole, ketoconazole, fluconazole, clotrimazole, isavuconazonium, miconozale, econazole, sulconazole, oxiconazole, a pharmaceutically acceptable salt thereof or a mixture thereof.

13. The composition according to claim 1 wherein said azole antifungal agent is fluconazole.

14. The composition according to claim 1 further comprising an effective amount of an additional antifungal agent.

15. The composition according to claim 14 wherein said additional antifungal agent is nystatin, amphotericin, terbinafine, anidulafungin, caspofungin, micafungin, flucytosine, griseofulvin, pentamine, oil of oregano, tea tree oil, caprylic acid, tumeric/curcumin or a mixture thereof.

16. A method of treating a fungal infection in a patient or subject in need comprising administering to said patient or subject an antifungal effective amount of an inhibitor compound of CDR1 and/or CDR2 in combination with an azole antifungal agent.

17. The method according to claim 16 wherein said inhibitor compound is a compound which is set forth in FIG. 1, FIG. 2, FIG. 3, FIG. 4, FIG. 5, FIG. 6, FIG. 7, FIG. 8, FIG. 9, FIG. 11, FIG. 12, FIG. 13, FIG. 15, FIG. 16, FIG. 17, FIG. 18, FIG. 19 hereof, or a pharmaceutically acceptable salt thereof or mixture thereof.

18. The method according to claim 16 wherein said inhibitor compound is a compound as set forth in FIG. 5, FIG. 7 or FIG. 9 hereof.

19. The method according to claim 16 wherein said compound is 1324.245, 2222.274, 2227.003, 2227.035, 2227.013, a mixture thereof or a pharmaceutically acceptable salt thereof.

20. The method according to claim 16 wherein said inhibitor compound is 1324.245, 1324.276, 1324.279, 2220-208, 2220-216, 2220-238, 2222-237, 2222-238, 2222-276, 2227-201 or a pharmaceutically acceptable salt, diastereomer, enantiomer, solvate, polymorph or a mixture thereof.

21. The method according to claim 16 wherein said inhibitor compound is a compound according to the chemical structure 1324: 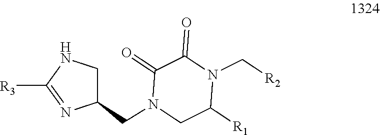
Where R₁ is H, C₁-C₁₂ (often C₁-C₆ or C₁-C₃) alkyl (optionally substituted with 1-3 halogen groups F, Cl, Br or I groups or 1-2 hydroxyl groups), naphthyl, 2-naphthylmethyl, 4-hydroxybenzyl, pyridin-3-yl-methyl, phenyl (optionally substituted with from 1-3 halogen groups or 1-2 hydroxyl groups), benzyl (optionally substituted with from 1-3 halogen groups and/or 1-2 hydroxyl groups) or pyridine-3-yl-methyl (often R₁ groups include S-2-naphthylmethyl, R-2-naphthylmethyl, S-4-hydroxybenzyl, S-pyridin-3-yl-methyl, S-benzyl, gamma-butyl, S-isobutyl, S-propyl, S-2-butyl, R-methyl, R-propyl, R-phenyl, S-tert-butyl, S-isopropyl, S-butyl, R-benzyl, R-2-butyl, R-pyridin-3-yl-methyl, S-4-fluoro-benzyl, R-isopropyl, S-cyclohexyl, R-butyl, S-methyl, R-isobutyl or R-tert-butyl);
R₂ is H, C₁-C₁₂ (often C₁-C₆ or C₁-C₃) alkyl (optionally substituted with 1-3 halogen groups F, Cl, Br or I groups or 1-2 hydroxyl groups) or —(CH₂)ₙphenyl (optionally substituted with 1-3 halogen groups, 1-2 hydroxyl groups, 1-3 —O—C₁-C₃) alkyl groups, and/or 1-3 optionally substituted C₁-C₄ alkyl groups which are optionally substituted with 1-3 halogen or 1-2 hydroxyl groups) (R₂ groups often include 2-(3-bromo-phenyl)-ethyl, m-tolylethyl, 2-(3-fluoro-phenyl)-ethyl, isobutyl, 2-Bicyclo[2.2.1]hept-2-yl-ethyl, cyclohexyl-butyl, cyclobutyl-methyl, n-butyl, ethyl, phenylpropyl,4-methyl-1-cyclohexylmethyl, cycloheptyl-methyl, 2-phenylbutyl, 2-(3,5-bis-trifluoromethyl-phenyl)-ethyl, 2-(4-Isobutyl-phenyl)-propyl, 3-cyclopentyl-propyl, cyclohexyl-methyl, (+/−)-2-Methylbutyl, cyclopentyl-methyl, heptyl, 2-(3-methoxy-phenyl)-ethyl, 4-phenylbutyl, 3-(3,4-dimethoxy-phenyl)-propyl, 3-methylbutyl or phenethyl);
R₃ is H, C₁-C₂₀, often C₁-C₁₂ (often C₁-C₆ or C₁-C₃) alkyl (optionally substituted with 1-3 halogen groups F, Cl, Br or I groups or 1-2 hydroxyl groups), C₂-C₆ alkene, often a vinyl group which is optionally substituted, —(CH₂)ₙphenyl (optionally substituted with 1-3 halogen groups, 1-2 hydroxyl groups, 1-3 —O—C₁-C₃) alkyl groups, and/or 1-3 optionally substituted C₁-C₄ alkyl or alkene groups which are optionally substituted with 1-3 halogen or 1-2 hydroxyl groups), —(CH₂)-biphenyl (optionally substituted with 1-3 halogen groups, 1-2 hydroxyl groups, 1-3 —O—C₁-C₃) alkyl groups, and/or 1-3 optionally substituted C₁-C₄ alkyl or alkene groups which are optionally substituted with 1-3 halogen or 1-2 hydroxyl groups) with the proviso that R1, R₂ and R₃, are not each simultaneously H, preferably no more than one of R1, R₂ and R₃ is H (R₃ groups often include 3-bromo-benzyl, 4-fluoro-phenyl, 4-methyl-cyclohexyl, 3-methyl-phenyl, tert-butyl, Bicyclo[2.2.1]hept-2-yl-methylcyclopentyl, ethyl, cyclobutyl, 4-methoxy-benzyl, 2-cyclohexyl-ethyl, cycloheptyl, propyl, biphenyl-4-yl, 2-(3,4-dimethoxy-phenyl)-ethyl, biphenyl-4-yl-methyl, 3,4-dimethoxy-benzyl, 4-bromo-benzyl, 1-ethyl-pentyl, 2-(2-trifluoromethyl-phenyl)-vinyl, 2-cyclopentyl-ethyl, hexyl, 3-fluoro-benzyl, benzyl or 3-phenyl-propyl; and n is 0-6, often 0-4, or a pharmaceutically acceptable salt, stereoisomer, diastereomer, enantiomer, solvate or polymorph thereof.

22. The method according to claim 16 wherein said inhibitor compound is a compound according to the chemical structure 2220 or 2222: 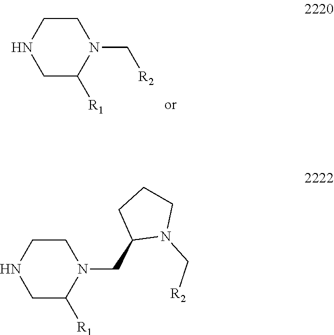
Where R₁ is H, C₁-C₁₂ (often C₁-C₆ or C₁-C₃) alkyl (optionally substituted with 1-3 halogen groups F, Cl, Br or I groups or 1-2 hydroxyl groups), naphthyl, optionally substituted with from 1-3 C₁-C₃ alkyl groups, pyridine, optionally substituted with 1 or 2 C₁-C₃ alkyl groups,
—(CH₂)ₙphenyl (optionally substituted with from 1-3 halogen groups, 1-2 hydroxyl groups or 1-3 C₁-C₃ alkyl groups, which are optionally substituted with from 1-3 halogen, often fluoro), (often R₁ is R-3-methylpyridine, S-3-methylpyridine, R-hydroxy-methyl, S-1-hydroxy-ethyl, R-isobutyl, S-hydroxy-methyl, S-4-hydroxybenzyl, R-propyl, R-1-hydroxy-ethyl, S-propanol, S-4-fluorophenyl-methyl, R-methyl, S-methyl, R-butyl, S-isopropyl, R-4-fluorophenyl-methyl, S-propyl, R-2-butyl, R-cyclohexyl-methyl, R-isopropyl, S-2-butyl, S-dimethyl, R-4-chlorophenyl-methyl or R-2-methylnaphthalene);
R₂ is C₁-C₁₂ (often C₁-C₆ or C₁-C₃) alkyl (optionally substituted with 1-3 halogen groups F, Cl, Br or I groups or 1-2 hydroxyl groups), a thiophenyl, furanyl or indolyl group each of which is optionally substituted with from 1-3 C₁-C₃ alkyl groups, —(CH₂)ₙphenyl (optionally substituted with from 1-3 halogen groups, 1-2 hydroxyl groups or 1-3 —O—C₁-C₃ alkyl or 1-3 C₁-C₃ alkyl groups, which are optionally substituted with from 1-3 halogen, often fluoro groups) (R₂ is often 2-thiophene-methyl, m-tolylethyl, 2-(3-methoxy-phenyl)-ethyl,
2-(3-trifluoromethyl-phenyl)-ethyl, 2-methylfuran, 2-(3-fluoro-phenyl)-ethyl, 2-methylbutyl,
m-xylene, 4-methyl-benzyl, heptyl, butyl, t-butylmethyl, 4-methoxy-benzyl, (1-phenyl-cyclopropyl)-methyl, p-tolylethyl, 4-ethyoxyphenylethyl, dicyclohexyl-ethyl, 3-indolylethyl
2-(4-methoxy-phenyl)-ethyl, cyclohexyl-ethyl, cyclohexyl-butyl, 3,4-dichlorophenethyl,
phenethyl, cyclohexyl-methyl or toluene); and n is 0-6, often 0-4, or a pharmaceutically acceptable salt, stereoisomer, diastereomer, enantiomer, solvate or polymorph thereof.

23. The method according to claim 22 wherein said inhibitor compound is according to the chemical structure 2220: 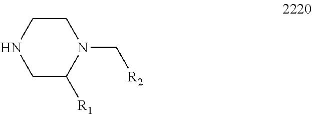
or
a pharmaceutically acceptable salt, diastereomer, enantiomer, solvate or polymorph thereof.

24. The method according to claim 22 wherein said inhibitor compound is according to the chemical structure 2222: 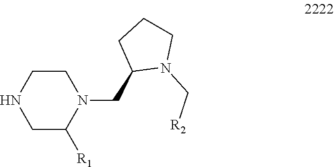
or
a pharmaceutically acceptable salt, diastereomer, enantiomer, solvate or polymorph thereof.

25. The method according to claim 16 wherein said inhibitor compound is a compound according to the chemical structure 2227: 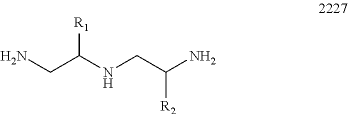
Where R₁ and R₂ are each independently H, C₁-C₁₂ (often C₁-C₆ or C₁-C₃) alkyl (optionally substituted with 1-3 halogen groups F, Cl, Br or I groups, 1-2 hydroxyl groups or an amine group which is optionally substituted with one or two methyl groups), naphthyl optionally substituted with 1-3 C₁-C₃ alkyl groups, pyridine, thiophene or imidazole, each of which is optionally substituted with 1 or 2 C₁-C₃ alkyl groups, C₂-C₆ thioether, naphthyl, optionally substituted with from 1-3 C₁-C₃ alkyl groups, —(CH₂)ₙphenyl (optionally substituted with from 1-3 halogen groups, 1-2 hydroxyl groups, 1-3 —O—C₁-C₃ alkyl groups or 1-3 C₁-C₃ alkyl groups, which are optionally substituted with from 1-3 halogen, often fluoro), analine, optionally substituted with from 1-3 C₁-C₃ alkyl groups, with the proviso that R₁ and R₂ are not both H (often R₁ and R₂ are each independently S-3-(methyl)-propylamine, S-methyl,
S-cyclohexyl-methyl, S-4-(methyl)butylamine, R-3-methylpyridine, dimethyl, R-2-methylnaphthalene, R-methyl-imidazolyl, S-isopropyl, R-isobutyl, R-2-methylthiophene, S-propyl, R-propyl, S-butyl, S-2-methylthiophene, R-1-hydroxy-ethyl, R-isopropyl, R-2-(methylthio)ethyl, S-4-ethoxybenzyl, R-2-butyl, R-methyl, R-4-methylanaline, R-4-ethoxybenzyl, S-ethyl or R-ethanol; and n is 0-6, often 0-4, or a pharmaceutically acceptable salt, stereoisomer, diastereomer, enantiomer, solvate or polymorph thereof.

26. The method according to claim 16 wherein said azole voriconazole, itraconazole, posaconazole, ketoconazole, fluconazole, clotrimazole, isavuconazonium, miconozale, econazole, sulconazole, oxiconazole, cyproconazole, defnoconazole, etaconazole, fenbuconazole, flusilazole, hexaconazole, propiconazole, tebuconazole, triademenol, uniconazole, epoxiconazole or a pharmaceutically acceptable salt or mixture thereof.

27. The method according to claim 16 wherein said azole antifungal agent is selected from the group consisting of voriconazole, itraconazole, posaconazole, ketoconazole, fluconazole, clotrimazole, isavuconazonium, miconozale, econazole, sulconazole, oxiconazole, a pharmaceutically acceptable salt thereof or a mixture thereof.

28. The method according to claim 16 wherein said fungal infection is azole antifungal agent resistant.

29. The method according to claim 16 wherein said fungal infection is an infection of Candida spp., Aspergillus spp Tricophyton spp., Histoplasma spp., Blastomyces spp., Paracoccidioides spp., Cryptococcus spp., Pneumocystis spp. (especially P. jirovecii), Mucor spp., Taloromyces spp., Sporothrix spp., Rhizopus spp., Absidia spp.

30. The method according to claim 16 wherein said fungal infection is an infection of Candida spp. or Aspergillus spp.

31. The method according to claim 29 wherein said fungal infection is an infection of C. albicans, C. krusei, C. rugosa, C. glabrata, C. parapsilosis, C. tropicalis, C. dubliniensis and C. auris.

32. The method according to claim 16 wherein said azole antifungal agent is fluconazole.

33. The method according to claim 16 wherein said inhibitor compound and said azole antifungal agent are further co-administered with an additional antifungal agent.

34. The method according to claim 33 wherein said additional antifungal agent is nystatin, amphotericin, terbinafine, anidulafungin, caspofungin, micafungin, flucytosine, griseofulvin, pentamine, oil of oregano, tea tree oil, caprylic acid, tumeric/curcumin or a mixture thereof.
